(19) United States
(12) Patent Application Publication
(10) Pub. No.: US 2021/0348213 A1
(43) Pub. Date: Nov. 11, 2021

(54) METHODS AND REAGENTS FOR ANALYSING NUCLEIC ACIDS FROM SINGLE CELLS

(71) Applicant: AUTOLUS LIMITED, London (GB)

(72) Inventors: Biao Ma, London (GB); Preeta Datta, London (GB); Yuchen Bai, London (GB); Shimobi Onuoha, London (GB); Martin Pulé, London (GB)

(21) Appl. No.: 17/284,098

(22) PCT Filed: Oct. 10, 2019
(86) PCT No.: PCT/GB2019/052882

§ 371 Date: Apr. 9, 2021

(30) Foreign Application Priority Data
Oct. 10, 2018 (GB) 1816522.5

(51) Int. Cl. C12Q 1/6806; C12Q 1/6869

(52) U.S. Cl. CPC C12Q 1/6806; C12Q 2600/158; C12Q 1/6869

(57) ABSTRACT

The present invention relates to a partition library which comprises a plurality of partitions which are useful for the analysis of the transcriptional response of a CAR to a target antigen. Further, the present invention relates to assays for the analysis of the transcriptional response of a CAR to a target antigen. The present invention also relates to kits comprising the plurality of partitions.

### FIELD OF THE INVENTION

The present invention relates to reagents and assays for analysing nucleic acids from single cells. The reagents and assays are useful for identifying chimeric antigen receptors having desirable properties.

### BACKGROUND TO THE INVENTION

Chimeric antigen receptors (CARs) are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

CAR T-cell therapies have demonstrated remarkable overall response rates in cancer patients. However, there are still challenges that preclude CAR T-cell from achieving their full therapeutic potential. One of these challenges is tumour antigen escape. Another challenge involves serious adverse events resulting from the activation of CAR T-cells, such as severe cytokine release syndrome (CRS) or neurotoxicity. Other challenges include poor T cell persistence and development of T cell exhaustion, which translates into lack of overall efficacy. While these mechanisms are not yet completely understood, it is clear that the molecular design of CARs is likely to have a strong influence upon them.

One major challenge for formulating CARs stems from their multi-components nature, each component being chosen from a selection of variants. Combining various components can result in a large pool of CARs where each unique combination can have a significant impact on the CAR-T cell biology and functionality. For instance, different binding domains derived from mAbs and targeting different epitopes on the tumour antigen can affect the killing of tumour cells and levels of cytokine release by the corresponding CAR-T cells. Binding domains with different binding kinetics to a given epitope on the target can also affect the signal transduction of CARs. Moreover, with the same binding domain, CARs with various spacers, transmembrane domains, and signalling endodomains can possess different functionalities that influence the biology and efficacy of modified T cells. A method to rapidly screen a large pool of CARs to identify the most effective and safest formulation is highly desirable in the field.

Methods in use at the moment focus on changes in protein expression following exposure to target antigen, such as cytokine secretion, which is commonly detected by ELISA or ELISpot, expression of T-cell phenotype, exhaustion markers, activation markers, and proliferation markers, and the killing of target cells, which are all generally detected by flow cytometry. The problem with these assays is that proteins are evaluated one-by-one and they are limited to cell-surface markers and secreted proteins. Another limitation lies in the fact that these assays can be performed on a limited number of CAR candidates at a time. These assays may additionally involve removing the target cells or washing before the analysis, which are likely to distort the true picture.

WO 2017/040694 describes one approach that allows the screening of large numbers of CARs using barcoded nucleic acids encoding CAR libraries. However, this method is restricted to the detection of changes in the phenotype.

Accordingly, there is a need in the art for an improved method for the direct interrogation of the CAR T-cell that allows detecting any marker, not only cell surface and secreted markers, and multiplexing, such that a large number of factors and CAR/CAR-T cell combinations can be evaluated at the same time.

### SUMMARY OF ASPECTS OF THE INVENTION

The inventors have developed reagents and methods to evaluate genes that are differentially expressed as a transcriptional response of CAR T− cells upon binding their cognate antigen. This analysis can be performed in a swift and straightforward manner. The present invention is particularly advantageous because it enables the direct acquisition of the nucleotide sequences forming part of the CAR-T cell transcriptome in a manner which is susceptible to automation. Key advantages include the direct and unequivocal identification of the particular CAR expressed by the T cell, which allows the direct comparison of T cells expressing different CARs under different conditions.

Additionally, the reagents and methods described herein overcome the limitations of the prior art as they permit the detection of any protein marker, i.e. intracellular as well as membrane-bound and secreted. Multiplexing is also possible and, with this, consistency may be achieved by mixing donors and intra-donor variability may be determined.

Thus, in a first aspect, the invention provides a partition library comprising a plurality of partitions, wherein each partition contains a single cell and a unique barcode molecule, wherein each cell comprises a cassette comprising a sequence encoding a chimeric antigen receptor (CAR) and a labelling sequence, wherein each CAR and each labelling sequence in the partition library are different.

In a particular embodiment, the labelling sequence is located in the 5′ untranslated region (UTR) of the sequence encoding the CAR.

In another particular embodiment, the labelling sequence is located in the sequence encoding the signal peptide of the sequence encoding the CAR.

In another particular embodiment, the labelling sequence is located in the 3′ UTR of the sequence encoding the CAR.

In another particular embodiment, the labelling sequence comprises at least 5 bp.

In another particular embodiment, each cassette further comprises a second sequence encoding a second CAR.

In another particular embodiment, each cassette further comprises a third sequence encoding a third CAR.

In another particular embodiment, the plurality of cassettes are DNA or RNA.

In another particular embodiment, the cells are cytolytic immune cells. In a preferred embodiment, the cytolytic immune cells are T cells or NK cells.

In another particular embodiment, the cells are incubated with a target cell expressing a target antigen.

In a second aspect, the invention provides an assay for analysing the transcriptional response of a CAR-expressing cell to a target antigen, which comprises the following steps:
(i) providing a plurality of partitions according to the invention;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) identifying genes within a given set which are differentially expressed by the cell following exposure to target antigen

In a particular embodiment, step (vi) identifies at least one gene selected from the group consisting of a gene related to cytokine production, a gene encoding a marker specific of a subset of T cells or NK cells, a gene encoding a marker of T cell or NK cell exhaustion, a gene encoding a marker of T cell or NK cell activation, a gene encoding a marker of T cell or NK cell proliferation, and a gene encoding a marker of T cell or NK cell killing.

In a third aspect, the invention provides an assay for comparing the transcriptional responses of a plurality of cells to a target antigen, which comprises the following steps:
(i) providing a plurality of partitions according to the invention, each cell in the partition expressing a different CAR against the same target antigen;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) comparing the expression of genes between sequence sets.

In a particular embodiment, step (vi) further identifies at least one gene selected from the group consisting of a gene related to cytokine production, a gene encoding a marker specific of a subset of T cells or NK cells, a gene encoding a marker of T cell or NK cell exhaustion, a gene encoding a marker of T cell or NK cell activation, a gene encoding a marker of T cell or NK cell proliferation, and a gene encoding a marker of T cell or NK cell killing.

In a fourth aspect, the invention provides a kit comprising a partition library according to the first aspect of the invention and at least one reagent suitable to carry out the assays according to the invention.

In a particular embodiment, the kit further comprises one or more components selected from the group consisting of partitioning fluids, barcode molecule libraries, which may be associated or not with microcapsules (e.g. beads), reagents for disrupting cells, reagents for amplifying nucleic acids, and any other component required to carry out the assay of the invention.

In another particular embodiment, the kit further comprises instructions for using the kit according to the assay according to the fourth aspect of the invention.

### DESCRIPTION OF THE FIGURES

FIG. 1. Schematic representation showing the structure of a standard CAR.

FIG. 2. Schematic representation depicting the method for identifying CARs with desirable properties.

FIG. 3. Determination by flow cytometry of the expression level of peripheral blood mononuclear cells (PBMCs) transduced with different vectors having the sequence encoding for RQR8 and an anti-CD19 CAR. Each vector was labelled with the Barcode 10 sequence at different positions of the 5′ UTR.

FIG. 4. Sequencing results revealed that the Barcode 10 sequence is in the transcript derived from the anti-CD29 CAR constructs. The sequences of Barcode 10 is shown in bold; the Kozak sequence is shown underlined; and the RQR8 coding sequence is highlighted in grey.

FIG. 5. Transduction Efficiencies. Representative FACS plot demonstrating transduction efficiency of lentiviral constructs carrying the anti-CD19 scFv CAT-19, FMC63, or HD37; or a scFv against the avian influenza virus H5N1. Upper panels: PBMCs transduced with the individual barcoded lentiviral constructs were stained with APC-conjugated QBEND10 (α-RQR8 APC in the figure), and anti-idiotype antibodies against either CAT-19, FMC63, or HD37 (all fluorochrome-conjugated). No anti-idiotype antibody was available for H5N1. Cells were then stained with an appropriate PE-conjugated secondary antibody. Middle panels: A background staining for the secondary antibody was also performed where cells were stained only with APC-conjugated QBEND10, and the appropriate PE-conjugated secondary antibody. Lower panels: A background staining for QBEND10 was performed where non-transduced (NT) PBMCs of matching donors were stained with APC-conjugated QBEND10, and anti-idiotype antibodies against either CAT-19, FMC63, or HD37, followed by staining with a PE-conjugated appropriate secondary antibody.

FIG. 6: FACS-based Killing Assay of Target cells by Anti-CD19 CAR T cells. CAT-19, FMC63, HD37, and H5N1 (control) barcoded CAR T cells, as well as non-transduced (NT) T cells from the same donor, were cultured either alone (No target), at a 1:1 ratio with SupT1 NT (Sup-T1), or at a 1:1 ratio with SupT1 NT cells modified to express CD19 (Sup-T1-CD19); in the absence of cytokine support for 72 h. a) Representative FACS plots demonstrating the killing ability of anti-CD19 CAR T cells. Cells from each group were harvested and stained with PE-conjugated anti-CD2 and PECy7-conjugated anti-CD3 antibodies. Double-positive events are effector T cells (CAR T cells); double-negative events are Target cells. b) Graph depicting the percentage of target cells remaining after 72 h in culture with the indicated effector T cells. c) and d) Cytokine production by effector cells in co-culture with target cells. Supernatant from the co-culture of effector T cells and target cells described above was harvested at 72 h and assayed by ELISA for c) IL-2 and d) IFN-γ levels.

FIG. 7: Quantifying cDNA derived from the amplification of the transcriptome of individual CAR T cells. CAT-19, FMC63, HD37, and H5N1 (control) barcoded CAR T cells, cultured either alone or co-cultured with SupT1-CD19 target cells for 72 h, were pooled into two groups: a) SupT1-CD19-treated and b) Non-treated. Each group was partitioned into single cells and all cellular mRNA reversed transcribed into cDNA and amplified. The quality (graphs) and quantity (tables) of the cDNA were analysed on a Tapestation. Peaks on the left and on the right corresponding to 25 bp and 1500 bp, respectively, are molecular weight markers. The central peak corresponds to cDNA generated from the transcriptome of single-cells, which is approximately _{˜}450 bp in size.

FIG. 8: tSNE plot of CAR-expressing T cells with CD19 stimulation (CAR) (i.e. co-incubated with SupT-1 CD19+ cells) versus CAR-expressing T cells without the stimulation (NT).

FIG. 9: tSNE plot of cells transduced with H5N1 CAR (H5N1) vs cells transduced with CAT19 CAR following co-incubations with SupT-1 CD19+ cells.

### DETAILED DESCRIPTION OF THE INVENTION

The inventors have developed assays and reagents for identifying chimeric antigen receptors (CARs) with desirable properties. The assay exploits the use of a barcoded DNA cassette library which encodes CARs combined with the partition of single cells, which allows the direct identification of the particular CAR in a population of CAR-T cells expressing different CARs. The resulting data can be used to obtain the transcriptomic signature of each particular CAR-T cell. Advantageously, the assay is susceptible to automation and does not require the manual manipulation of samples to isolate single CAR-T cells.

### 1. Partition Library

In a first aspect, the present invention relates to a partition library which comprises a plurality of partitions, hereinafter “the partition library of the invention”, wherein each partition contains a single cell and a unique barcode molecule, wherein each cell comprises a cassette comprising a sequence encoding a chimeric antigen receptor (CAR) and a labelling sequence, wherein each CAR and each labelling sequence in each partition of the partition library are different.

### 1.1. Partition

The term “partition”, as used herein, refers to discrete compartments or partitions, which are used indistinctly herein. Each partition maintains a separation of its own contents from the contents of other partitions. A partition may be a droplet, macrovesicle, or a vessel. When the partitions refer to a droplet, they may comprise an aqueous fluid within a non-aqueous continuous phase, for example, an oil phase. When the partitions refer to a macrovesicle, it has an outer barrier surrounding an inner fluid centre or core, or, in some cases, they may comprise a porous matrix that is capable of entraining and/or retaining materials within its matrix. When the partitions refer to a container or vessel, these may be wells, microwells, tubes, vials, through ports in nanoarray substrates, for example, BioTrove nanoarrays, or other containers.

The partitions described herein may comprise small volumes, such as less than 10 μL, less than 5 μL, less than 1 μL, less than 500 nL, less than 100 nL, less than 50 nL, less than 10 nL, less than 5 nL, less than 1 nL, less than 900 picoliters (pL), less than 800 pL, less than 700 pL, less than 600 pL, less than 500 pL, less than 400 pL, less than 300 pL, less than 200 pL, less than 100 pL, less than 50 pL, less than 20 pL, less than 10 pL, less than 1 pL, or even less. Alternatively or in combination, the partitions may be of uniform size or heterogeneous size, with a diameter less than 1 mm, less than 500 μm, less than 250 μm, less than 100 μm, less than 90 μm, less than 80 μm, less than 70 μm, less than 60 μm, less than 50 μm, less than 40 μm, less than 30 μm, less than 20 μm, less than 10 μm, or less than 5 μm, or at least about 1 μm.

The partition library of the invention comprises a plurality of partitions, each partition containing a single cell and a unique barcode.

The term “partitioning”, as used herein, refers to the compartmentalisation, depositing or partitioning individual cells into distinct compartments or partitions. Any method for partitioning a population cells into individual cells, i.e. by controlling the occupancy of the resulting partitions (i.e. number of cells per partition), is suitable for the purposes of the present invention. These include, without limitation, the use of techniques based on microfluidic networks, droplets, microwell plates, and automatic collection of cells using capillaries, magnets, an electric field, or a punching probe. Partitioning of cells can be conveniently carried out using commercially available instruments, such as the ddSEQ Single-Cell Isolator, by Bio-Rad (Hercules, Calif., USA) and Illumina, (San Diego, Calif., USA), the Chromium system, by 10× Genomics (Pleasanton, Calif., USA), the Rhapsody Single-Cell Analysis System, by Becton, Dickinson and Company (BD, Franklin Lakes, N.J., USA), the Tapestri Platform (MissionBio, San Francisco, Calif., USA).

In order to ensure that those partitions that are occupied are primarily occupied by a single cell, it may be desirable that partitions contain less than one cell per partition. Thus, the majority of occupied partitions may include no more than one cell per occupied partition. In some cases, the partitioning process is conducted such that fewer than 25%, fewer than 20%, fewer than 15%, fewer than 10%, fewer than 5%, or fewer than 1% of the occupied partitions contain more than one cell.

### 1.2. Unique Barcode

Each partition contains a single cell and a unique barcode molecule. The term “barcode” or “barcode molecule”, as used herein, refers to a sequence, a label, or identifier that can be part of an analyte to convey information about the analyte. Barcodes can allow for identification and/or quantification of individual sequencing-reads in real time. The barcode is unique in the sense that all the barcodes in one partition are the same, but the barcodes in each partition are different from each other. Thus, in operation, the same barcode will be incorporated to all the cDNA products that are obtained by RT-PCR in a single cell. A barcode can be a sequence tag attached to an analyte (e.g. nucleic acid molecule) or a combination of the tag in addition to an endogenous characteristic of the analyte (e.g. size of the analyte or end sequence). Barcodes can have a variety of different formats, for example, barcodes can include: polynucleotide barcodes; random nucleic acid and/or amino acid sequences; and synthetic nucleic acid and/or amino acid sequences. A barcode can be attached to an analyte in a reversible or irreversible manner. The barcode can be added to, for example, a fragment of a deoxyribonucleic acid (DNA) or ribonucleic acid (RNA) sample before, during, and/or after sequencing of the sample. The barcode may be generated in a combinatorial manner. Barcodes that may be used with methods of the present disclosure are described in, for example, US Patent Pub. No. 2014/0378350.

The barcode molecule may be a polynucleotide. The length of a polynucleotidic barcode molecule may be at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250 nucleotides, at least 500 nucleotides, or longer.

The structure of the barcode oligonucleotides may include a number of sequence elements useful in the processing of the nucleic acids from the co-partitioned cells in addition to the oligonucleotide barcode sequence. These sequences include targeted or random/universal amplification primer sequences for amplifying the genomic DNA from the individual cells within the partitions while attaching the associated barcode sequences, sequencing primers or primer recognition sites, hybridisation or probing sequences, e.g. for identification of presence of the sequences or for pulling down barcoded nucleic acids, or any of a number of other potential functional sequences.

One example of a barcode oligonucleotide for use in RNA analysis is coupled to a bead by a releasable linkage, such as a disulfide linker. The oligonucleotide may include functional sequences that are used in subsequent processing. As will be appreciated, the functional sequences may be selected to be compatible with a variety of different sequencing systems, such as 454 Sequencing, Ion Torrent Proton or PGM, Illumina X10, etc., and the requirements thereof. A barcode sequence is included within the structure for use in barcoding the sample RNA. An mRNA specific priming sequence, such as poly-T sequence may also be included in the oligonucleotide structure. Other sequences may be used as primer sequences in the context of the present invention, including, without limitation, a sequence which is complementary to a region of a sequence encoding one of the IgG variable domains.

An anchoring sequence segment may be included to ensure that the poly-T sequence hybridises at the sequence end of the mRNA. This anchoring sequence can include a random short sequence of nucleotides, e.g., 1-mer, 2-mer, 3-mer or longer sequence, which will ensure that the poly-T segment is more likely to hybridise at the sequence end of the poly-A tail of the mRNA.

An additional sequence segment may be provided within the oligonucleotide sequence. In some cases, this additional sequence may provide a unique molecular identifier (UMI) sequence segment, such as a random sequence (for example, a random N-mer sequence) that varies across individual oligonucleotides coupled to a single partition, whereas the barcode sequence may be constant among oligonucleotides tethered to an individual partition. This UMI serves to provide a unique identifier of the starting mRNA molecule that was captured, in order to allow quantitation of the number of original expressed RNA. As will be appreciated, individual partitions may include tens to hundreds of thousands or even millions of individual oligonucleotide molecules, where the barcode molecule may be constant or relatively constant for a given partition, but where the UMI will vary across an individual partition. This UMI sequence segment may include from 5 to about 8 or more nucleotides within the sequence of the oligonucleotides. In some cases, the UMI sequence segment may be 2, 3, 4, 5, 6, 7, 8, 9, 10, 11, 12, 13, 14, 15, 16, 17, 18, 19 or 20 nucleotides in length or longer.

In some cases, it may be desirable to incorporate multiple different barcodes within a given partition, either attached to a single or multiple beads within the partition. For example, in some cases, a mixed, but known barcode sequences set may provide greater assurance of identification in the subsequent processing, e.g., by providing a stronger address or attribution of the barcodes to a given partition, as a duplicate or independent confirmation of the output from a given partition.

The oligonucleotides may be releasable from the beads upon the application of a particular stimulus to the beads. In some cases, the stimulus may be a photo-stimulus, e.g. through cleavage of a photo-labile linkage that releases the barcode molecule. In other cases, a thermal stimulus may be used, where elevation of the temperature of the beads environment will result in cleavage of a linkage or other release of the barcode molecule from the beads. In still other cases, a chemical stimulus is used that cleaves a linkage of the oligonucleotides to the beads, or otherwise results in release of the barcode molecule from the beads, such as through exposure to a reducing agent, e.g. DTT.

The barcode is delivered to a partition via a bead. The term “bead”, as used herein, refers to a microparticle having a diameter of between 1 μm and 1 mm, irrespective of the precise interior or exterior structure. Non-limiting examples of beads include a microcapsule and a microsphere. The bead may be porous, non-porous, solid, semi-solid, semi-fluidic, or fluidic. The bead may be dissolvable, disruptable, or degradable. The bead may not be degradable. The bead may be a gel bead. The gel bead may be a hydrogel bead. The gel bead may be formed from molecular precursors, such as a polymeric or monomeric species. A semi-solid bead may be a liposomal bead. A solid bead may comprise metals including iron oxide, gold, and silver. The bead may be a silica bead. The bead may be rigid. In some cases, the bead may be flexible and/or compressible.

Beads may be of uniform size or heterogeneous size. In some cases, the diameter of a bead may be less than 1 mm, less than 500 μm, less than 250 μm, less than 100 μm, less than 90 μm, less than 80 μm, less than 70 μm, less than 60 μm, less than 50 μm, less than 40 μm, less than 30 μm, less than 20 μm, less than 10 μm, or less than 5 μm, or at least about 1 μm.

Beads may be of uniform size or heterogeneous size. In some cases, the diameter of a bead may be at least about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm.

Any suitable number of barcode molecules can be associated with a bead such that the barcoded molecules are present in the partition at a predefined concentration. Such predefined concentration may be selected to facilitate certain reactions for generating a sequencing library, such as amplification, within the partition. The population of beads may provide a diverse barcode sequence library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences.

Methods for connecting a barcode molecule to a bead are known in the art.

As will be appreciated, the above-described occupancy rates are also applicable to partitions that include both cells and additional reagents, including, without limitation, microcapsules or beads carrying barcoded oligonucleotides. At least 5%, at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or at least 100% of the partitions contain both a microcapsule comprising barcode molecules and a cell.

In addition to microcapsules or beads, other reagents may also be co-partitioned with the cells. The cells may be partitioned along with lysis reagents in order to release the contents of the cells within the partition. In such cases, the lysis agents can be contacted with the cell suspension concurrently with, or immediately prior to the introduction of the cells into the partitioning junction/droplet generation zone. Examples of lysis agents include bioactive reagents, such as lysis enzymes, for example, lysozymes, achromopeptidase, lysostaphin, labiase, kitalase, lyticase, and a variety of other commercially available lysis enzymes. Other lysis agents may additionally or alternatively be co-partitioned with the cells to cause the release of the cell's contents into the partitions. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g. non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. This stimulus may be the same as the stimulus described elsewhere herein for release of oligonucleotides from their respective bead (e.g. microcapsule). Alternatively, this may be a different and non-overlapping stimulus, in order to allow an encapsulated cell to be released into a partition at a different time from the release of barcode molecule into the same partition.

In some cases, it may be desirable to keep the barcode molecule attached to the bead (e.g. microcapsule). For example, the partition-bound oligonucleotides may be used to hybridise and capture the mRNA on the solid phase of the partition in order to facilitate the separation of the RNA from other cell contents.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides, also referred to herein as “switch oligos” or “template switching oligonucleotides”, which can be used for template switching. Switching can be used to increase the length of a cDNA. Template switching can be used to append a predefined nucleic acid sequence to the cDNA. In one example of template switching, cDNA can be generated from reverse transcription of a template, e.g. cellular mRNA, where a reverse transcriptase with terminal transferase activity can add additional nucleotides, e.g., polyC, to the cDNA in a template independent manner.

The additional reagents may be delivered to a partition by means of additional beads, or together with the barcode molecules.

### 1.3. Cassette

As used herein, the term “cassette” refers to one or more nucleic acid sequences which comprise a coding sequence, and which have been constructed in such a way so as to facilitate addition of the cassette to a vector. Additionally, the cassettes described herein facilitate incorporation of additional sequences in operable linkage with the prepared cassette sequences for preparation of desired CAR sequences, e.g., in one or two cloning steps.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the sequences being linked are contiguous and, where necessary to join two protein coding regions, contiguous and in reading frame.

The partition library of the invention comprises a plurality of cassettes, i.e. it comprises at least two, or at least three, or at least four, or at least five, or at least 6, or at least 7, or at least 8, or at least 9, or at least 10, or at least 20, or at least 30, or at least 40, or at least 50, or at least 100, or more cassettes.

The plurality of cassettes that form part of the partition library according to the invention may be DNA or RNA cassettes.

### 1.4. Chimeric Antigen Receptor (CAR)

In the partition library of the invention, each cell comprises a cassette comprising a sequence encoding a CAR and a labelling sequence, wherein each CAR and each labelling sequence in the partition library are different.

The term “chimeric antigen receptor” or “CAR” or “chimeric T cell receptor” or “artificial T cell receptors” or “chimeric immunoreceptors”, as used herein, refers to a chimeric type I trans-membrane protein which connects an extracellular antigen-recognising domain (binder) to an intracellular signalling domain (endodomain). The binder is typically a single-chain variable fragment (scFv) derived from a monoclonal antibody (mAb), but it can be based on other formats which comprise an antigen binding site. A spacer domain is usually necessary to separate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

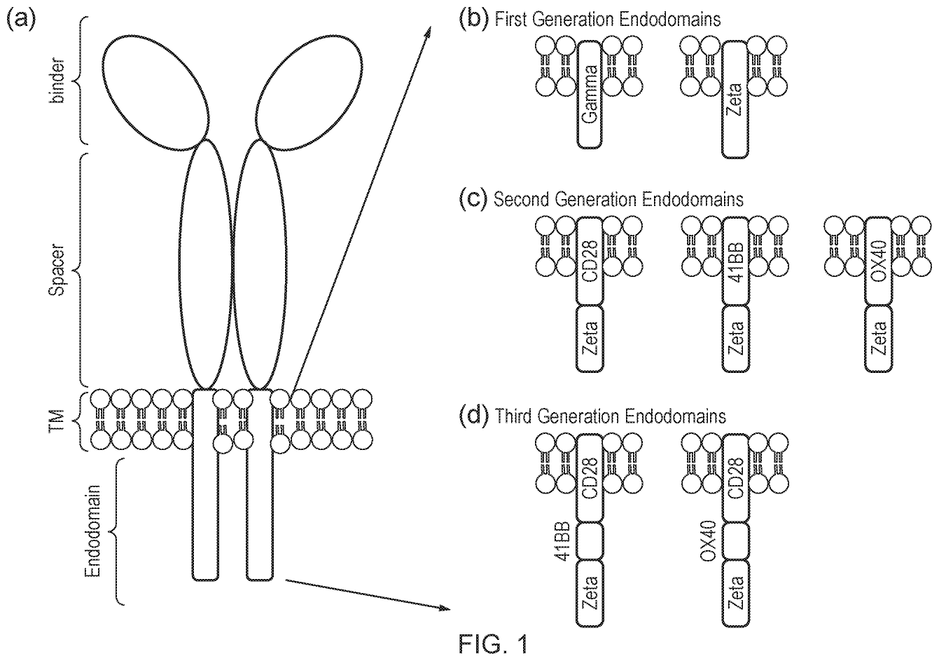

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals. Even more potent third generation CARs have now been described which have endodomains capable of transmitting activation, proliferation and survival signals. CARs typically therefore comprise: (i) an antigen-binding domain; (ii) a spacer; (iii) a transmembrane domain; and (iii) an intracellular domain which comprises or associates with a signalling domain (see FIG. 1).

A CAR may have the general structure:
Antigen binding domain-spacer domain-transmembrane domain-intracellular signalling domain (endodomain).

When the CAR binds the target antigen, this results in the transmission of an activating signal to the T-cell it is expressed on. Thus the CAR directs the specificity and cytotoxicity of the T cell towards cells expressing the targeted antigen.

In the partition library of the invention, the CARs encoded by the sequence comprised in each cassette may all have the same binding specificity or different binding specificities. Advantageously, the CAR encoded in each cassette may be different. For example, where all the CARs encoded in the partition library share the same sequence except for the antigen-binding domain, and all the antigen-binding domains have the same binding specificity, the library will be suitable to test differences in the intracellular signalling derived from the different antigen binding domain.

Virtually, any target may be used for the purposes of this invention. Targets that are specific to a particular condition or disease are particularly useful. Non-limiting examples of suitable targets include, without limitation, CD19, CD20, CD21, CD22, CD33, CD38, CD45, CD52, CD79a, CD79b, CEA, GD2, BCMA, HER2, HER3, EGFR, PD-1, PD-L1, TACI, FcRH5, ROR1, and DLL3.

In an embodiment, the target of the antigen binding domain of the CAR is CD19. In another embodiment, the target of the antigen binding domain of the CAR is CD20. In another embodiment, the target of the antigen binding domain of the CAR is CD21. In another embodiment, the target of the antigen binding domain of the CAR is CD22. In another embodiment, the target of the antigen binding domain of the CAR is CD33. In another embodiment, the target of the antigen binding domain of the CAR is CD38. In another embodiment, the target of the antigen binding domain of the CAR is CD45. In another embodiment, the target of the antigen binding domain of the CAR is CD52. In another embodiment, the target of the antigen binding domain of the CAR is CD79a. In another embodiment, the target of the antigen binding domain of the CAR is CD79b. In another embodiment, the target of the antigen binding domain of the CAR is CEA. In another embodiment, the target of the antigen binding domain of the CAR is GD2. In another embodiment, the target of the antigen binding domain of the CAR is BCMA. In another embodiment, the target of the antigen binding domain of the CAR is HER2. In another embodiment, the target of the antigen binding domain of the CAR is HER3. In another embodiment, the target of the antigen binding domain of the CAR is EGFR. In another embodiment, the target of the antigen binding domain of the CAR is PD-1. In another embodiment, the target of the antigen binding domain of the CAR is PD-L1. In another embodiment, the target of the antigen binding domain of the CAR is TACI. In another embodiment, the target of the antigen binding domain of the CAR is FcRH5. In another embodiment, the target of the antigen binding domain of the CAR is ROR1. In another embodiment, the target of the antigen binding domain of the CAR is DLL3.

Different antigen binding domains having the same binding specificity may be used in each of the cells comprised in the plurality of partitions of the partition library of the invention. For example, where the target is CD19, one cell comprised in the plurality of partitions may comprise a cassette having a sequence encoding a CAR comprising an antigen binding domain derived from fmc63 antibody, and another cell may comprise a cassette having a sequence encoding a CAR comprising an antigen binding domain derived from 4G7 antibody, and another cell may comprise a cassette having a sequence encoding a CAR comprising an antigen binding domain derived from SJ25C1 antibody, and another cell may comprise a cassette having a sequence encoding a CAR comprising an antigen binding domain derived from HD37 antibody, and another cell may comprise a cassette having a sequence encoding a CAR comprising an antigen binding domain derived from CAT19 antibody (as described in WO2016/139487), and another cell may comprise a cassette having a sequence encoding a CAR comprising an antigen binding domain derived from CD19ALAb antibody (as described in WO2016/102965).

The antigen binding domain may be selected from a scFv, a dAb or a Fab.

The antigen binding domain may be a protein that binds to the target antigen, such as a protein receptor or a ligand.

The CARs encoded by the sequence comprised in each cassette may all have the same spacer domain or different spacer domains. For example, where all the CARs encoded in the plurality of cassettes comprised in each cell of the partition library have the same sequence except for the spacer domain, the library will be suitable to test differences in the intracellular signalling derived from the different spacer domain.

Virtually, any spacer which serves to separate the binder from the membrane and to allow it adopt a suitable orientation may be used for the purposes of this invention. Non-limiting examples of suitable spacers include, without limitation, the Fc region of IgG1, the Fc region of IgM, the stalk region of CD8a, the stalk region of CD28, and the IgG1 hinge region.

The CARs encoded by the sequence comprised in each cassette may all have the same transmembrane domain or different transmembrane domains. For example, where all the CARs encoded in the plurality of cassettes have the same sequence except for the transmembrane domain, the library will be suitable to test differences in the intracellular signalling derived from the different transmembrane domain.

Virtually, any transmembrane domain which anchors the CAR protein in the cell membrane and connects the spacer to the endodomain may be used for the purposes of this invention. Non-limiting examples of suitable transmembrane domains include, without limitation, the transmembrane domain derived from CD28, CD8a or TYRP-1.

The CARs encoded by the sequence comprised in each cassette may all have the same endodomain or different endodomains. For example, where all the CARs encoded in the plurality of cassettes have the same sequence except for the endodomain, the library will be suitable to test differences in the intracellular signalling derived from the different endodomain.

Virtually, any endodomain which anchors the CAR protein in the cell membrane and connects the spacer to the endodomain may be used for the purposes of this invention. The most commonly used endodomain component is that of CD3ζ which contains 3 ITAMs. This transmits an activation signal to the T cell after antigen is bound. CD3ζ may not provide a fully competent activation signal and additional co-stimulatory signalling may be needed. Non-limiting examples of co-stimulatory domains that can be used with CD3ζ to transmit a proliferative/survival signal include the endodomains from CD28, OX40, 4-1BB, CD27, and ICOS.

In another embodiment, each cassette in the plurality of cassettes comprised in each cell of the partition library may further comprise a second sequence encoding a second CAR. In a particular embodiment, each cassette may further comprise a third sequence encoding a third CAR. It will be appreciated that the invention also contemplates partition libraries where each cassette comprises more than three sequences encoding more than three CARs. The CARs in cassettes containing more than one sequence encoding a CAR may have the same or different binding specificity. These are particularly useful when testing LOGIC gates.

“Logic-gated” CAR pairs which, when expressed by a cell, such as a T cell or NK cell, are capable of detecting a particular pattern of expression of at least two target antigens. If the at least two target antigens are arbitrarily denoted as antigen A and antigen B, the three possible options are as follows:

“OR GATE”—T cell or NK cell triggers when either antigen A or antigen B is present on the target cell;

“AND GATE”—T cell or NK cell triggers only when both antigens A and B are present on the target cell;

“AND NOT GATE”—T cell or NK cell triggers if antigen A is present alone on the target cell, but not if both antigens A and B are present on the target cell.

The skilled person will be able to make the necessary changes to the spacer and/or endodomains of the logic gated CAR pairs as required to render the logic gate functional.

Examples of logic-gated chimeric antigen receptor pairs are described in WO2015/075468, WO2015/075469, and WO2015/075470.

Advantageously, there may be a nucleic acid sequence located between each of the sequences encoding a CAR in this embodiment, hereinafter “coexpr”, which enables co-expression of the different CARs as separate entities. Where there are more than one coexpr, these may be the same or different.

Coexpr may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al., 2001, J Gen Virol 82:1027-41). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donnelly et al., 2001, as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001, as above).

The cleavage site may comprise the 2A-like sequence shown as SEQ ID NO: 1 (RAEGRGSLLTCGDVEENPGP).

### 1.5. Additional Sequences

Each cassette may further comprise a sequence encoding a marker gene. The term “marker gene”, as used herein, refers to a gene that enables measurement of transduction efficiency and allows purification of transduced cells. Examples of marker genes include, without limitation, Neomycin resistance, truncated nerve growth factor receptor, ΔEGFR and truncated CD34.

Each cassette may further comprise a sequence encoding a suicide gene. The term “suicide gene”, as used herein, refers to a gene that facilitates deletion of T-cells in case of toxicity. Non-limiting examples of marker genes include Herpes Simplex Virus thymidine kinase (HSVtk), inducible Caspase 9, inducible FAS, CD20, Δc-myc, ΔEGFR, and human thymidylate kinase.

Each cassette may further comprise a sequence encoding a marker-suicide gene, such as RQR8, which is composed of two rituximab binding epitopes flanking the QBEnd10 epitope on a CD8 stalk which enables selection with the cliniMACS CD34 system and deletion through both CDC and ADCC with rituximab (Philip et al., 2014, Blood 124:1277-87).

The CAR(s), suicide gene, marker gene and/or marker-suicide gene of each cassette of the present invention may comprise a signal peptide so that when the proteins are expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the SEQ ID NO: 2 to 4 or a variant thereof having 5, 4, 3, 2 or 1 amino acid mutations (insertions, substitutions or additions) provided that the signal peptide still functions to cause cell surface expression of the protein.

SEQ ID NO: 2:
MGTSLLCWMALCLLGADHADG

The signal peptide of SEQ ID NO: 2 is compact and highly efficient. It is predicted to give about 95% cleavage after the terminal glycine, giving efficient removal by signal peptidase.

SEQ ID NO: 3:
MSLPVTALLLPLALLLHAARP

The signal peptide of SEQ ID NO: 3 is derived from IgG1.

SEQ ID NO: 4:
MAVPTQVLGLLLLWLTDARC

The signal peptide of SEQ ID NO: 4 is derived from CD8.

The signal peptide for the first CAR may have a different sequence from the signal peptide of the second CAR (and from the subsequence CARs), and from the other coding sequences.

Each cassette may further comprise a 5′ untranslated region (UTR) upstream of the sequence encoding the CAR or, where applicable, the first coding sequence of the cassette. The 5′ UTR, or leader sequence or leader RNA, is the region of an mRNA that is directly upstream from the initiation codon which contains the Kozak consensus sequence (ACCAUGG; SEQ ID NO: 5). Its size is dependent upon the particular promoter used and it may range from about 30 bp to more than 1 kbp.

Each cassette may further comprise a 3′ UTR downstream of the sequence encoding the CAR or, where applicable, the last coding sequence of the cassette. The 3′ UTR is the section of mRNA that immediately follows the translation termination codon. The 3′-UTR contains the sequence AAUAAA that directs addition of several hundred adenine residues called the poly(A) tail to the end of the mRNA transcript.

Each cassette may further comprise a 5′ UTR and a 3′ UTR as described previously.

### 1.6. Labelling Sequence

Each cassette of the plurality of cassettes comprised in each cell of the partition library library of the invention comprises a sequence encoding a CAR and a labelling sequence, wherein each CAR and each labelling sequence in the partition library is different.

The term “labelling sequence” or “labelling sequence tag”, as used herein, refers to a random nucleotide sequence, such as a random N-mer sequence, which is different in each of the cassettes of the partition library. This unique labelling sequence serves to provide a unique identifier of the CAR which is encoded by a sequence in the cassette.

When used in the method according to the invention, the labelling sequence provides a label on individual CAR transcripts. The labelling sequence is reversed transcribed and incorporated into the pool of cDNA originating from said cell thus readily allowing the identification of the cDNA sequence encoding each CAR in the plurality of cDNA molecule.

This unique labelling sequence may include from 5 to about 500 or more nucleotides within the sequence of oligonucleotides. The labelling sequence segment can be at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500 nucleotides in length or longer.

The labelling sequence may consist of between 5 and 500 nucleotides, or between 10 and 400 nucleotides, or between 15 and 300 nucleotides, or between 20 and 200 nucleotides, or between 30 and 100 nucleotides, or between 40 and 90 nucleotides, or between 50 and 80 nucleotides in length.

Examples of labelling sequences include, without limitation, the following sequences:

(SEQ ID NO: 6)
5′-GCTGGCACTACGACA-3′
(SEQ ID NO: 7)
5′-GACATTATCTTTCGC-3′
(SEQ ID NO: 8)
5′-CATTTTACCTACCTG-3′
(SEQ ID NO: 9)
5′-CACAATATTGTTGGG-3′
(SEQ ID NO: 10)
5′-ATTGCCTTGGCATCT-3′
(SEQ ID NO: 11)
5′-CGATTCTAGTGACGA-3′
(SEQ ID NO: 12)
5′-CAAGACAAACGATGC-3′
(SEQ ID NO: 13)
5′-CAACTACAGTTTCAC-3′
(SEQ ID NO: 14)
5′-GCGCTAGTCTCCACA-3′
(SEQ ID NO: 15)
5′-TCCACTATCGTTCAA-3′

The sequences shown with SEQ ID NO: 6-15 are derived from Saccharomyces cerevisiae AGA1 gene, but other labelling sequences may be used.

The labelling sequence may be located in the 5′ UTR upstream of the sequence encoding the CAR or, where appropriate, upstream of the first coding sequence comprised in each cassette. The number of base pairs between the labelling sequence and the Kozak sequence may be 0 bp, or at least 1 bp, or at least 2 bp, or at least 3 bp, or at least 4 bp, or at least 5 bp, or at least 6 bp, or at least 7 bp, or at least 8 bp, or at least 9 bp, or at least 10 bp, or at least 11 bp, or at least 12 bp, or at least 13 bp, or at least 14 bp, or at least 15 bp, or at least 16 bp, or at least 17 bp, or at least 18 bp, or at least 19 bp, or at least 20 bp, or at least 21 bp, or at least 22 bp, or at least 23 bp, or at least 24 bp, or at least 25 bp, or at least 26 bp, or at least 27 bp, or at least 28 bp, or at least 29 bp, or at least 30 bp, or at least 35 bp, or at least 40 bp, or at least 45 bp, or at least 50 bp, or at least 60 bp, or at least 70 bp, or at least 80 bp, or at least 90 bp, or at least 100 bp, or at least 150 bp, or at least 200 bp, or at least 300 bp, or at least 400 bp, or at least 500 bp, or at least 600 bp, or at least 700 bp, or at least 800 bp, or at least 900 bp, or at least 1,000 bp, or more.

The labelling sequence may be located in the 3′ UTR downstream of the sequence encoding the CAR or, where appropriate, downstream of the last coding sequence comprised in each cassette. The number of bp between the labelling sequence and the stop codon may be 0 bp, or at least 1 bp, or at least 2 bp, or at least 3 bp, or at least 4 bp, or at least 5 bp, or at least 6 bp, or at least 7 bp, or at least 8 bp, or at least 9 bp, or at least 10 bp, or at least 11 bp, or at least 12 bp, or at least 13 bp, or at least 14 bp, or at least 15 bp, or at least 16 bp, or at least 17 bp, or at least 18 bp, or at least 19 bp, or at least 20 bp, or at least 21 bp, or at least 22 bp, or at least 23 bp, or at least 24 bp, or at least 25 bp, or at least 26 bp, or at least 27 bp, or at least 28 bp, or at least 29 bp, or at least 30 bp, or at least 35 bp, or at least 40 bp, or at least 45 bp, or at least 50 bp, or at least 60 bp, or at least 70 bp, or at least 80 bp, or at least 90 bp, or at least 100 bp, or at least 150 bp, or at least 200 bp, or at least 300 bp, or at least 400 bp, or at least 500 bp, or at least 600 bp, or at least 700 bp, or at least 800 bp, or at least 900 bp, or at least 1,000 bp, or more. Alternatively, the number of bp between the labelling sequence and the polyadenylation signal sequence (e.g. AAUAAA; SEQ ID NO: 16) may be 0 bp, or at least 1 bp, or at least 2 bp, or at least 3 bp, or at least 4 bp, or at least 5 bp, or at least 6 bp, or at least 7 bp, or at least 8 bp, or at least 9 bp, or at least 10 bp, or at least 11 bp, or at least 12 bp, or at least 13 bp, or at least 14 bp, or at least 15 bp, or at least 16 bp, or at least 17 bp, or at least 18 bp, or at least 19 bp, or at least 20 bp, or at least 21 bp, or at least 22 bp, or at least 23 bp, or at least 24 bp, or at least 25 bp, or at least 26 bp, or at least 27 bp, or at least 28 bp, or at least 29 bp, or at least 30 bp, or at least 35 bp, or at least 40 bp, or at least 45 bp, or at least 50 bp, or at least 60 bp, or at least 70 bp, or at least 80 bp, or at least 90 bp, or at least 100 bp, or at least 150 bp, or at least 200 bp, or at least 300 bp, or at least 400 bp, or at least 500 bp, or at least 600 bp, or at least 700 bp, or at least 800 bp, or at least 900 bp, or at least 1,000 bp, or more.

Alternatively, the labelling sequence may be located in the sequence encoding the signal peptide of the sequence encoding the CAR or, where appropriate, in the sequence encoding the signal peptide of the first coding sequence in each cassette. In this embodiment, the labelling sequence may be constructed by making synonymous mutations in the sequence encoding the signal peptide. The labelling sequence will depend on the sequence of the particular signal peptide and it will comprise part or the entire sequence encoding the signal peptide. The labelling sequence may be obtained by making a synonymous mutation in at least one nucleotide, or in at least two nucleotides, or in at least three nucleotides, or in at least four nucleotides, or in at least five nucleotides, or in at least 6 nucleotides, or in at least 7 nucleotides, or in at least 8 nucleotides, or in at least 9 nucleotides, or in at least 10 nucleotides, or in at least 11 nucleotides, or in at least 12 nucleotides, or in at least 13 nucleotides, or in at least 14 nucleotides, or in at least 15 nucleotides, or in at least 16 nucleotides, or in at least 17 nucleotides, or in at least 18 nucleotides, or in at least 19 nucleotides, or in at least 20 nucleotides, or in at least 21 nucleotides, or in at least 22 nucleotides, or in at least 23 nucleotides, or in at least 24 nucleotides, or in at least 25 nucleotides, or in at least 30 nucleotides, or in at least 40 nucleotides, or in at least 50 nucleotides, or in at least 60 nucleotides, or more of the nucleotide sequence encoding the signal peptide.

### 1.7. Cell

The present invention provides a plurality of partitions, wherein each partition contains a single cell and a unique barcode molecule, wherein each cell comprises a cassette comprising a sequence encoding a CAR and a labelling sequence, wherein each CAR and each labelling sequence in the partition library are different.

Each cell in the plurality of partitions may contain at least one different cassette. Thus, each cell in the plurality of cells may contain one, two, three or more different cassettes.

The cassette or cassettes may be introduced into a plurality of host cells so that they express the CAR(s) and, where applicable, the additional sequences using a vector. In some embodiments, each cell may contain at least one different vector. Thus, each cell in the plurality of cells may contain one, two, three or more different vectors.

The vector may, for example, be a plasmid or a viral vector, such as a retroviral vector or a lentiviral vector, or a transposon-based vector or synthetic mRNA.

The vector may be capable of transfecting or transducing a cell.

In embodiments where each cassette comprises two or more sequences encoding two or more CARs, each cell in the plurality of cells may comprise two or more CARs. For example it may comprise a double or triple OR gate, or an AND gate, or an AND NOT gate.

“Logic-gated” chimeric antigen receptor pairs which, when expressed by a cell, such as a cytolytic cell (e.g. a T cell or NK cell), are capable of detecting a particular pattern of expression of at least two target antigens. If the at least two target antigens are arbitrarily denoted as antigen A and antigen B, the three possible options are as follows:

“OR GATE”—cytolytic cell triggers when either antigen A or antigen B is present on the target cell;

“AND GATE”—cytolytic cell triggers only when both antigens A and B are present on the target cell;

“AND NOT GATE”—cytolytic cell triggers if antigen A is present alone on the target cell, but not if both antigens A and B are present on the target cell.

The cells may be cytolytic immune cells, such as T cells or NK cells. The plurality of cells may be T cells. Alternatively, the plurality of cells may be NK cells.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumour cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The plurality of cells may be Natural Killer cells (or NK cells). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The plurality of cells according to the invention may either be created ex vivo either from the peripheral blood from a single subject, or from the peripheral blood from a number of different subjects.

Alternatively, the plurality of according to the invention may be derived from ex vivo differentiation of inducible progenitor cells or embryonic progenitor cells to cells, such a cytolytic cells. Alternatively, an immortalised cytolytic cell line which retains its lytic function and could act as a therapeutic may be used.

In all these embodiments, chimeric polypeptide-expressing cells are generated by introducing the cassette library or plurality of vectors according to the invention by one of many means, including transduction and transfection.

The cell of the invention may be an ex vivo cell from a subject. The cell may be from a peripheral blood mononuclear cell (PBMC) sample. Cells may be activated and/or expanded prior to being transduced with the cassette library or plurality of vectors according to the invention, for example by treatment with an anti-CD3 monoclonal antibody.

The plurality of cells contained in the plurality of partitions of the partition library of the invention need not be pure. Thus the plurality of described herein may contain at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 96%, at least 97%, at least 98%, at least 99%, or 100% of cells as described herein.

It may be particularly advantageous to use a population that has been enriched in cells as described herein. This may be performed by various methods that are conventional in the art, such as FACS or using magnetic beads. The skilled person will readily know which cell surface antigens that are suitable for the enrichment.

The plurality of cells may be obtained using peripheral blood obtained from a single subject or peripheral blood obtained from a number of different subjects. It will be appreciated that the former permits the evaluation of intra-donor variability, while the latter provides consistency.

In an embodiment the cells may be incubated with a target cell expressing the target antigen. Alternatively, the cells may have been incubated with a target cell expressing the target antigen.

This may be attained by co-incubating the target cell expressing at least one target antigen specific for the CAR(s) expressed by the plurality of cells and, optionally, shaking gently to facilitate the cells coming into contact.

The incubation step may be carried out in a liquid medium. Any liquid media suitable for the culture of peripheral blood mononuclear cells (PBMCs) cells may be used including, without limitation, RPMI 1640 medium (ThermoFisher), AIM-V medium (ThermoFisher), OpTmizer medium (ThermoFisher), Human Blood Cell Medium (Cell Applications, Inc.), R5 medium (RPMI 1640 with 5% human serum, 55 μM 2-mercaptoethanol, 2 mM L-glutamine, 100 U/ml penicillin, 100 μg/ml streptomycin, 10 mM HEPES, 1 mM sodium pyruvate and 1% MEM nonessential amino acids), and a medium containing RPMI 1640, 5% fetal calf serum, 2 mM L-glutamine, 1% penicillin/streptomycin, and 50 μM β-mercaptoethanol.

Any cell expressing any target antigen may be used for the purposes of this invention as long as the CAR(s) expressed by the plurality of cells is specific for said target antigen. Targets that are specific to a particular condition or disease are particularly useful. Non-limiting examples of suitable target antigens include, without limitation, CD19, CD20, CD21, CD22, CD33, CD38, CD45, CD52, CD79a, CD79b, CEA, GD2, BCMA, HER2, HER3, EGFR, PD-1, PD-L1, TACI, FcRH5, ROR1, DLL3, and combinations thereof. The target cell may express the target antigen naturally. Alternatively, the target cell may be any cell that has been engineered to express a recombinant target antigen. Those skilled in the art can readily generate cells that express a recombinant target antigen, for example, by transducing a suitable cell line, e.g. SupT1, with an expression vector coding for the target antigen to achieve different levels (e.g. high, medium, low) of target antigen expression on the cell surface. It is generally considered that a high level of target antigen expression is higher than 10,000 copies of target antigen per cell, a medium level is between 1,000 and 10,000 copies of target antigen per cell, and a low lever is lower than 1,000 copies of target antigen per cell, although high, medium and low levels of target antigen expression on the cell surface depend upon the type of cell and the particular antigen.

Cells which do not express the target antigen or which have low levels of expression may be used as controls or reference samples for the target cell.

Different ratios of CAR-expressing cells to target cells, i.e. effector:target ratio, may be used, such as 1:1, 2:1, 3:1, 4:1, 5:1, 6:1, 7:1, 8:1, 9:1, 10:1, 15:1, 20:1, 50:1, 100:1, 250:1, 500:1, 1,000:1.

Where the plurality of cells comprises one CAR, the cells may express multiple molecules of a single type of CAR, this step will result in the activation of the cells and the killing of cognate target cells.

Where the plurality of CAR-expressing cells comprises two or more CARs forming the so-called “logic gates”, this step will result in the activation or inhibition of the cells according to the particular logic gate expressed and the pattern of target antigens expressed by the cognate target cells. The activation of the cells will result in the killing of cognate target cells, while the inhibition of the cells will result in the cognate target cells being spared.

The activation of CAR-expressing cells may be determined by any conventional method, such as by detecting secretion of IL-2 and/or INF-γ into the culture medium. Likewise, the killing of cognate target cells by the CAR-expressing cells may be determined by any method known in the art, such as the ⁵¹Chromium release assay or by flow cytometry assays.

After the co-incubation, the plurality of cells will contain cells which have been activated or, where appropriate, inhibited. Different CARs having the same specificity may affect the extent or degree of activation or, where appropriate, inhibition.

Optionally, the remaining target cells (if there are any remaining) may be removed from the plurality of CAR-expressing cells by any suitable method, such as, by cell sorting using magnetic particles.

### 2. Assay

The partition library of the invention described herein may be advantageously used in a method for identifying a CAR with certain properties that are particularly desirable. This may be achieved by detecting any changes in expression of genes in CAR-T cells

### 2.1. Assay for Analysing the Transcriptional Response of a CAR-Expressing Cell to a Target Antigen

Thus, in another aspect, the present invention relates to an assay for analysing the transcriptional response of a CAR to a target antigen, hereinafter “the first assay of the invention”, which comprises the following steps:
(i) providing a plurality of partitions according the invention;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) identifying genes within a given set which are differentially expressed by the cell following exposure to target antigen.

The terms “plurality of partitions according to the invention”, “cell”, “target antigen”, and “CAR” have been described in detail previously in the context of the first aspect of the invention and their definitions, particular features and embodiments apply equally to the first assay of the invention.

In a first step, the first assay of the invention comprises providing a plurality of partitions according to the invention.

In an embodiment, the cells may be or may have been incubated with a target cell expressing the target antigen, as previously described.

This may be attained by co-incubating the target cell expressing at least one target antigen specific for the CAR(s) expressed by the plurality of cells and, optionally, shaking gently to facilitate the cells coming into contact.

In a second step, the first assay of the invention comprises a step of performing reverse transcription such that all mRNA sequences in the cell within each of the partitions are barcoded with the unique barcode molecule.

The reverse transcriptase may be conveniently provided within the partition. The reverse transcription reaction may be performed using any commercially available reverse transcriptase according to conventional methods, which include a step of annealing and elongation.

The reverse transcription may be performed using an oligonucleotide that forms part of the barcode molecule as priming agent.

The primer portion of the barcode molecule can anneal to a complementary region of a cell's nucleic acid. Extension reaction reagents, e.g., DNA polymerase, nucleoside triphosphates, co-factors (e.g., Mg²⁺ or Mn²⁺), that are also co-partitioned with the cells and beads, then extend the primer sequence using the cell's nucleic acid as a template, to produce a complementary fragment to the strand of the cell's nucleic acid to which the primer annealed, which complementary fragment includes the oligonucleotide and its associated barcode sequence. Annealing and extension of multiple primers to different portions of the cell's nucleic acids will result in a large pool of overlapping complementary fragments of the nucleic acid, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow formation of a hairpin structure or partial hairpin structure, the reduces the ability of the molecule to be the basis for producing further iterative copies.

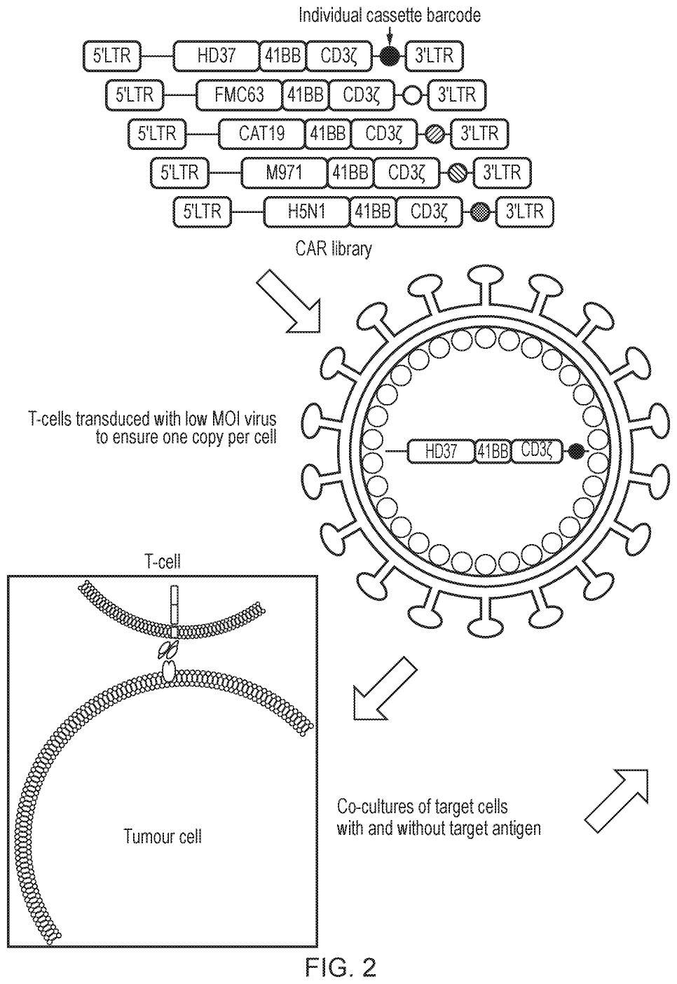

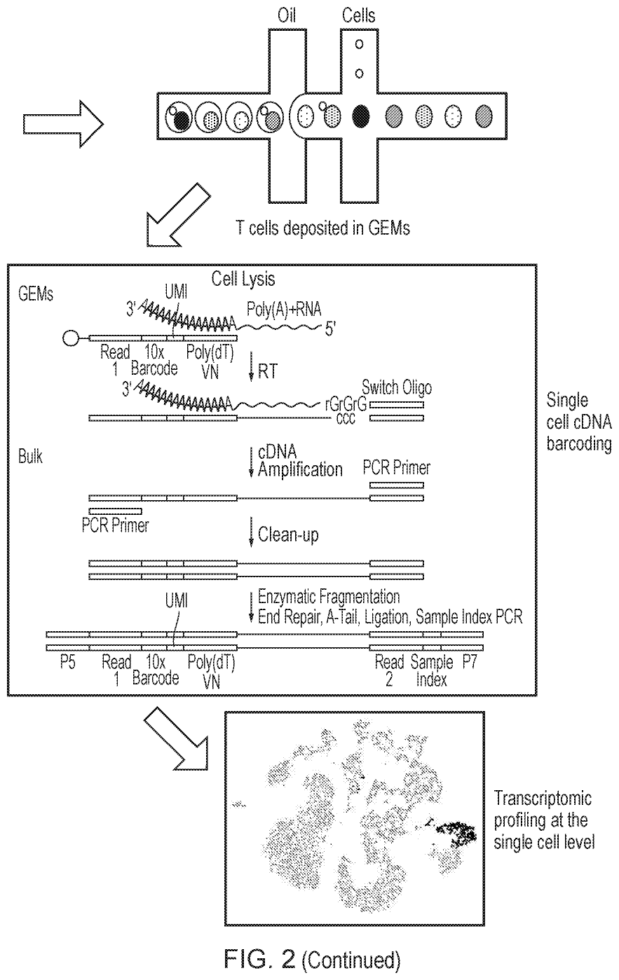

In operation, and with reference to FIG. 2, a cell according to the invention is co-partitioned along with a barcode bearing bead and lysed while the barcoded oligonucleotides are released from the bead. The poly-T portion of the released barcode oligonucleotide then hybridises to the poly-A tail of each mRNA molecule present in the cell. The poly-T segment then primes the reverse transcription of the mRNA to produce a cDNA transcript of the mRNA, but which includes each of the sequence segments of the barcode oligonucleotide. Again, because the oligonucleotide includes an anchoring sequence, it will more likely hybridise to and prime reverse transcription at the sequence end of the poly-A tail of the mRNA. Within any given partition, all of the cDNA transcripts of the individual mRNA molecules will include a common or unique barcode sequence segment. However, by including the unique random N-mer sequence, the transcripts made from different mRNA molecules within a given partition will vary at this unique sequence. This provides a quantitation feature that can be identifiable even following any subsequent amplification of the contents of a given partition, e.g., the number of unique segments associated with a common barcode can be indicative of the quantity of mRNA originating from a single partition, and thus, a single cell. As noted above, the transcripts are then amplified, cleaned up and sequenced to identify the sequence of the cDNA transcript of the mRNA, as well as to sequence the barcode segment and the unique sequence segment.

While a poly-T primer sequence is described, other targeted or random priming sequences may also be used in priming the reverse transcription reaction. In some cases, the primer sequence can be a 5′ UTR specific primer sequence which targets the specific 5′ UTR of the plurality of cassettes. In other cases, the primer sequence can be a gene specific primer sequence which targets specific genes for reverse transcription. Such target genes may comprise genes encoding the CAR components, particularly those encoding the VH and VL domains, genes related to cytokine production (e.g. genes encoding IL2, IFNγ), genes encoding markers of naïve or central memory T cells, genes encoding markers of effector/memory cells, genes encoding markers of exhaustion, and other genes encoding markers of activation, proliferation and killing. The sequences of these primers will be readily determined by the person skilled in the art.

Optionally, a step of amplification by polymerase chain reaction (PCR) may be performed prior to the disruption of the partitions and pooling of the barcoded nucleic acids with the purpose of enriching a subset of nucleic acids corresponding to the specific sequence where the labelling sequence according to the invention is located. The labelling sequence has been described in detail in the context of the first aspect of the invention and its particular embodiments apply equally to the assay of the invention. This amplification step may additionally amplify nucleic acids corresponding to specific sequences encoding the CAR components, genes related to cytokine production (e.g. IL2, IFNγ), markers of naïve or central memory T cells, markers of effector/memory cells, markers of exhaustion, and other markers of activation, proliferation and killing. One or more gene specific primers can be used together with the barcode molecule for primer extension using the cDNA molecule as a template. The sequences of these primers will be readily determined by the person skilled in the art. For example, the primers to amplify the specific sequence encoding the CAR of each cassette may comprise an oligonucleotide having a sequence complementary to the sequence encoding the endodomain component of the CAR, and an oligonucleotide having a sequence specific for the barcode molecule. The primers may conveniently be provided or delivered to the partition with a bead (e.g. microcapsule).

The amplification may be carried out for at least 5, at least 10, at least 15, at least 20, at least 25, at least 30, at least 40 or more cycles. In general, the amplification of the cell's nucleic acids is carried out until the barcoded overlapping fragments within the partition constitute at least 1× coverage of the particular portion or all of the cell's transcriptome, at least 2×, at least 3×, at least 4×, at least 5×, at least 10×, at least 20×, at least 40× or more coverage of the whole transcriptome or a relevant portion of interest.

Any of a variety of polymerases can be used in embodiments herein for primer extension, including, without limitation, exonuclease minus DNA Polymerase I large (Klenow) Fragment, Phi29 DNA polymerase, Taq DNA Polymerase, T4 DNA polymerase, T7 DNA polymerase, and the like. Further examples of polymerase enzymes that can be used in embodiments herein include thermostable polymerases. In some embodiments, a hot start polymerase is used. A hot start polymerase is a modified form of a DNA polymerase that can be activated by incubation at elevated temperatures.

As previously noted, each distinct labelling sequence according to the invention may correspond to each cell of the plurality of cells according to the invention. Enrichment increases accuracy and sensitivity of methods for sequencing immunoglobulin genes at a single cell level. Enrichment may lead to greater than or equal to 25%, 30%, 35%, 40%, 45%, 50%, 55%, 60%, 65%, 70%, 75%, 80%, 85%, 90%, 95% or more of total sequencing reads mapping to the enriched sequence.

The reverse transcription may be carried out by 5′ Rapid Amplification of cDNA Ends (5′-RACE). 5′-RACE, or “one-sided” PCR or “anchored” PCR, is a technique that facilitates the isolation and characterisation of 5′ ends from low-copy transcripts.

There are various systems available in the market that allow the automated generation of single cells transcriptomes. For example, single cell transcriptomes may be generated using a scRNA-seq microfluidics platform (10× Genomics).

Following the generation of barcoded template polynucleotides or derivatives (e.g. amplification products) thereof, subsequent operations may be performed, including enzymatic fragmentation, purification (e.g. via solid phase reversible immobilization (SPRI)) or further processing (e.g. shearing, addition of functional sequences, and subsequent amplification, e.g. by PCR). These operations may occur in bulk, for example, outside the partition.

In a third step, the first assay of the invention comprises a step of disrupting the partitions and pooling the barcoded nucleic acid sequences from the second step.

The partitions may be disrupted by any suitable means, such as by mechanical disruption, by an increase in pressure or by chemical disruption.

As will be understood, as a result of pooling the barcoded nucleic acid sequences from the second step, there is obtained a mixture of all of the cDNA transcripts of the individual mRNA molecules originally contained in the plurality of cells of the second step. Thus, there will be a mixture of unique barcode sequence segments, each identifying a different cell of origin.

Optionally, a step of amplification by PCR may be performed in bulk after pooling the barcoded nucleic acids in order to amplify nucleic acids corresponding to the specific sequence where the labelling sequence according to the invention is located. The labelling sequence has been described in detail in the context of the first aspect of the invention and its particular embodiments apply equally to the assay of the invention. This amplification step may additionally amplify nucleic acids corresponding to specific sequences encoding the CAR components, genes related to cytokine production (e.g. IL2, IFNγ), markers of naïve or central memory T cells, markers of effector/memory cells, markers of exhaustion, and other markers of activation, proliferation and killing. Optionally, one or more gene specific primers can be used together with the barcode molecule for primer extension using the cDNA molecule as a template. The sequences of these primers will be readily determined by the person skilled in the art. The primers may conveniently be provided or delivered to the partition with a bead (e.g. microcapsule).

The amplification may be carried out for at least 5, at least 10, at least 15, at least 20, at least 25, at least 30, at least 40 or more cycles. In general, the amplification of the cell's nucleic acids is carried out until the barcoded overlapping fragments within the partition constitute at least 1× coverage of the particular portion or all of the cell's transcriptome, at least 2×, at least 3×, at least 4×, at least 5×, at least 10×, at least 20×, at least 40× or more coverage of the transcriptome or its relevant portion of interest.

Any of a variety of polymerases can be used in embodiments herein for primer extension, including, without limitation, exonuclease minus DNA Polymerase I large (Klenow) Fragment, Phi29 DNA polymerase, Taq DNA Polymerase, T4 DNA polymerase, T7 DNA polymerase, and the like. Further examples of polymerase enzymes that can be used in embodiments herein include thermostable polymerases. In some embodiments, a hot start polymerase is used. A hot start polymerase is a modified form of a DNA polymerase that can be activated by incubation at elevated temperatures.

Enrichment increases accuracy and sensitivity of methods for sequencing individual genes at a single cell level.

In a fourth step, the first assay of the invention comprises a step of sequencing the pooled sequences.

The term “sequencing”, as used herein, refers to methods and technologies for determining the sequence of nucleotide bases in one or more polynucleotides. The polynucleotides can be, for example, deoxyribonucleic acid (DNA) or variants or derivatives thereof, such as single stranded DNA. DNA sequencing can be performed by any technique and system currently available, such as, next generation sequencing or high throughput sequencing techniques, including Roche 454 pyrosequencing and other sequencing technologies by Illumina, Pacific Biosciences, Oxford Nanopore, and Life Technologies.

It will be appreciated that several scRNA-seq methods may be used for the purposes of this invention. Non-limiting examples include the method described by Tang et al. (Tang et al., 2009, Nat Methods 6:377-82), the STRT method (Islam et al., 2011, Genome Res 21:1160-7), the SMART-seq method (Ramskold et al., 2012, Nat Biotechnol 30:777-82), the CEL-seq method (Hashimshony et al., 2012, Cell Rep 2:666-73), and the Quartz-seq method (Sasagawa et al. 2013, Genome Biol 14:R31). These protocols differ in terms of strategies for reverse transcription, cDNA synthesis and amplification, and the possibility to accommodate sequence-specific barcodes or the ability to process pooled samples. The present invention also contemplates the use future developments in the field of scRNA-seq. For example, the pooled RNA sequences were sequenced using the HiSeq 2×150 bp platform (HiSeq 2500 System, Illumina).

In a fifth step, the first assay of the invention comprises a step of analysing the pooled sequences with the same unique barcode.

The DNA sequences obtained from this step all contain a barcode. By assembling the sequences according to their barcode, sequences can be grouped according to their starting cell. The sets of sequences having the same unique barcode may comprise sequences forming the entire transcriptome of the plurality of cells of the second step. These sequences will include a sequence corresponding to the labelling sequence according to the invention.

In any given group of DNA sequences, the presence of a specific labelling sequence according to the invention indicates the particular CAR expressed by the cell. Where applicable, the presence of a specific labelling sequence according to the invention indicates the particular combination of CARs, marker genes, suicide genes, and marker-suicide genes expressed by the cell.

The analysis may also include further assembling sequences according to each UMI in order group sequences according to their starting mRNA molecule, then merging highly similar assembled sequences. This step allows quantitation of the number of original expressed RNA transcripts, i.e. quantitation of gene expression levels.

Once the sequences have been grouped by cell of origin, the particular CAR expressed by the cell has been identified, and, optionally, the mRNA molecules have been quantitated, the gene expression profile of individual cells may be further analysed. The analysis involves detecting differences in gene expression level.

These analyses may be done automatically using an appropriate software. For example, the bulk sequencing data may be demultiplexed using Illumina's bcl2fastq software according to the sample index associated with the individual sequencing library to yield RNA-seq data in FASTQ format. RNA-seq data may be then analysed using 10× Genomics software package Cell Ranger version 3.0.2 on a Linux server. Cell Ranger is a set of analysis pipelines that process the RNA-seq output to align reads, generate feature-barcode matrices based on detected 10× Barcodes associated with single cells and perform clustering and gene expression analysis. The Cell Ranger ‘count’ pipeline can take FASTQ files and perform single cell sequence analysis including alignment to reference genome and transcriptome profiles, filtering, barcode counting, and UMI counting. Cell Ranger ‘aggr’ is a pipeline used to aggregate the output of ‘count’ generated from separate pools into one result file with sample attributes defined for each pool. The type of file produced by ‘aggr’ bears the extension ‘cloupe’. Subsequently, 10× software Loupe Cell Browser 3.1.0 may be used to process the ‘cloupe’ file. Through this software, the 5′ gene expression profiles of individual CAR-T cells can be extracted from the entire dataset for further analysis. The Loupe Cell Browser allows the identification of significant genes by comparing the expression profiles among different cell population, and clustering of cell types based on their unique gene expression profiles. This software also generates tSNE plots using a dimensionality reduction algorithm to graphically visualize the separation of very large datasets, such as the separation of cell clusters.

The skilled person will appreciate that other suitable software(s) may be used in the analysis of the RNA sequencing data.

In a sixth step, the first assay of the invention comprises a step of identifying genes within a given set which are differentially expressed by the cell following exposure to target antigen.

Differences in gene expression may be determined by comparing the expression levels with reference values for each gene. Typically, the reference value for any given gene is the expression level of said gene in a reference sample. Conveniently, the entire transcriptome obtained in the seventh step may be compared with a standard or reference transcriptional signature.

A “reference sample” or “standard sample” or “reference transcriptional signature” or “standard transcriptional signature”, as used herein, refers to the pool of sequences of RNA transcripts or transcriptional signature obtained from a plurality of cells according to the invention with which a comparison wants to be drawn. For example, a reference sample may be obtained from a plurality of cells according to the invention which has been incubated in step (ii) with a cell which does not express the target antigen. In another example, a reference sample may be obtained from a plurality of cells according to the invention which has been incubated in step (ii) with a cell which expresses the target antigen at a low level. Both these examples of reference sample allow the investigation of the transcriptional changes that occur in the CAR-T cell upon contacting its cognate antigen. In another example, a reference sample may be obtained from a plurality of cells according to the invention which expresses a reference CAR, such as a CAR that is considered a gold standard for a given target and/or therapeutic indication, and which plurality of cells has been incubated in step (ii) with a cell which expresses the target antigen. This allows the examination of the responses of CAR-T cells expressing different CARs upon contacting the target antigen at the transcriptional level.

The profile of gene expression levels in the reference sample may be generated from a population of two or more individual cells expressing a CAR. The population, for example, may comprise 2, 3, 4, 5, 10, 15, 20, 30, 40, 50, 100, 500, 1,000, 10,000, 100,000 or more individual cells.

Once the gene expression levels in relation to reference values have been determined, it is necessary to identify if there are alterations in the expression of the genes, i.e. an increase or decrease of gene expression. The expression of a gene is considered to be increased when the expression levels increase with respect to the reference sample by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more. Similarly, the expression of a gene is considered decreased when its levels decrease with respect to the reference sample by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by at least 100% (i.e., absent).

The term “gene”, as used herein, refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. A gene can be a DNA sequence that encodes for an mRNA that encodes a protein. A gene may be comprised of a single exon and no introns, or can include multiple exons and one or more introns. One of two or more identical or alternative forms of a gene present at a particular locus is referred to as an “allele” and, for example, a diploid organism will typically have two alleles of a particular gene.

Genes may be identified from the sets of pooled sequences obtained in the seventh step by any conventional method, typically a method of sequence alignment. Methods for the alignment of sequences for comparison are well known in the art, such methods include GAP, BESTFIT, BLAST, FASTA and TFASTA. GAP uses the algorithm of Needleman and Wunsch (1970, J Mol Biol 48:443-53) to find the global (i.e. spanning the complete sequences) alignment of two sequences that maximizes the number of matches and minimizes the number of gaps. The BLAST algorithm (Altschul et al., 1990, J Mol Biol 215: 403-10) calculates percent sequence identity and performs a statistical analysis of the similarity between the two sequences. The software for performing BLAST analysis is publicly available through the National Centre for Biotechnology Information (NCBI), which compares a query sequence against a publicly available NCBI database, such as BLASTN or TBLASTX. Global percentages of similarity and identity may also be determined using one of the methods available in the MatGAT software package (Campanella et al., 2003, BMC Bioinformatics 4:29). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or over selected domains or conserved motif(s), using the programs mentioned above using the default parameters. For local alignments, the Smith-Waterman algorithm is particularly useful (Smith & Waterman, 1981, J Mol Biol 147:195-7).

Virtually, the change of expression of any gene may provide information about the transcriptional response of a CAR to a target antigen. Non-limiting examples of genes whose change of expression may provide information about the transcriptional response of a CAR to a target antigen include genes encoding markers of subsets of cells (e.g. naïve T cells, activated T cells, central memory T cells, effector memory T cells, NK cells), genes related to cytokine production (e.g. IL2, IFNγ), genes encoding markers of exhaustion, and other genes encoding markers of activation, proliferation and killing.

A naïve T cell (Th0 cell) is a mature T cell that has not encountered its cognate antigen. Naïve T cells are commonly characterized by the surface expression of L-selectin (CD62L) and C—C Chemokine receptor type 7 (CCR7); the absence of the activation markers CD25, CD44 or CD69; and the absence of memory CD45RO isoform. They also express functional IL-7 receptors, consisting of subunits IL-7 receptor-α, CD127, and common-γ chain, CD132.

Memory T cells are long-lived and can quickly expand to large numbers of effector T cells upon re-exposure to their cognate antigen. Memory T cells may be either CD4+ or CD8+ and usually express CD45RO. Memory T cell subtypes include central memory T cells (TCM cells), which express CD45RO, CCR7, L-selectin (CD62L), and also have intermediate to high expression of CD44; and effector memory T cells (TEM cells), which express CD45RO but lack expression of CCR7 and L-selectin, and also have intermediate to high expression of CD44.

Markers commonly used to monitor T cell exhaustion include, in respect of CD8+ T cells, PD-1, CTLA-4, LAG-3, TIM-3, 2B4/CD244/SLAMF4, CD160, TIGIT, IL-2 (loss of IL-2 production), TNF-α (impaired production), IFN-γ (impaired production), and CC(β) chemokines (impaired production), and Granzyme B (high levels); and in respect of CD4+ T cells, markers include PD-1, CTLA-4, LAG-3, TIM-3, 2B4/CD244/SLAMF4, CD160, TIGIT, IL-2 (loss of IL-2 production), TNF-α (impaired production), IFN-γ (impaired production), and CC(β) chemokines (impaired production), GATA-3, Bcl-6, Helios, CXCR5, ICOS, IL-4 (increased production), IL-6 (increased production), IL-21 (increased production), Bcl-6, IRF4, and STAT4.

Markers commonly used to monitor T cell activation include CD25, CD44, CD62L^{low}, and CD69 (all up-regulated).

Markers commonly used to monitor T cell proliferation include PCNA, Ki67, histone H3 pSer28, BrdU, VPD450, and MCM-2.

Markers commonly used to monitor T cell killing include CD8A, CD8B, EOMES, and perforin (PRF1).

Markers commonly used to monitor activation and exhaustion of NK cells include: CD56, killer-like immunoglobulin receptor (KIR) family, NGK2A, NGK2C, NGK2D, CD16, 2B4, NKp30, NKp44, NKp46, Fas, CD40L, TRAIL, INF-γ, TNF-α, CXCL8, Perforin, IL-7R-α, CXCR1, CXCR3, CXCR4, CCR7, and CX3CR1; markers of exhaustion only of NK cells include PD-1 and Tim3.

The combination of labelling sequence and barcode sequence is particularly advantageous because it allows for multiplexing and assaying several parameters, such as different cell subtypes, different levels of expression of CARs per cell, or intra-donor variability, in a single assay.

### 2.2. Assay for Comparing the Transcriptional Responses of a Plurality of Cells to a Target Antigen

The first assay of the invention may be adapted for the determination of differences in gene expression in cells expressing different CARs against the same antigen upon interacting with the target antigen. By combining the labelling sequence, which identifies each different CAR, with the barcode sequence, which identifies the transcript sequences expressed by each cell, it is possible to assay cells, each cell expressing a different CAR, for expression of many different genes in the same assay by multiplexing. This allows to determine the effect that different CARs, or CAR components, may have in the cells upon binding to its cognate target. For example, by varying the antigen-binding domain of the CAR while maintaining the antigen specificity, the effects of the different binding kinetics in the activation of the cell may be determined. In another example, by varying the endodomain of the CAR while maintaining the other components, the effects of the different combinations of intracellular signalling domains in cell activation, proliferation and/or killing may be determined.

Thus, in another aspect, the present invention relates to an assay for comparing the transcriptional responses of a plurality of cells to a target antigen, hereinafter “the second assay of the invention”, which comprises the following steps:
(i) providing a plurality of partitions according to the invention, the cell in each partition expressing a different CAR against the same target antigen;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) comparing the expression of genes between sequence sets obtained in step (vii).

The terms “plurality of cells”, “cell”, “target antigen”, and “CAR” have been described in detail previously in the context of other aspects of the invention and their definitions, particular features and embodiments apply equally to the second assay of the invention.

In a first step, the second assay of the invention comprises a step of providing a plurality of partitions according to the invention, the cell in each partition expressing a different CAR against the same target antigen.

In an embodiment, the cells may be or may have been incubated with a target cell expressing the target antigen, as previously described.

This may be attained by co-incubating the target cell expressing at least one target antigen specific for the CAR(s) expressed by the plurality of cells and, optionally, shaking gently to facilitate the cells coming into contact.

The CARs expressed by each cell all have the same target specificity but differ in one or more of the CAR components, i.e. the antigen binding domain, the spacer domain, the transmembrane domain, and/or the intracellular signalling domain.

Accordingly, the CARs expressed by each cell may comprise the same spacer, transmembrane and intracellular signalling domains and may differ in the antigen binding domain, provided that the different antigen binding domains have the same target specificity. Alternatively, the CARs expressed by each cell may comprise the same antigen binding domain, transmembrane and intracellular signalling domains and may differ in the spacer. Alternatively, the CARs expressed by each cell may comprise the same antigen binding domain, spacer and intracellular signalling domain and may differ in the transmembrane domain. Alternatively, the CARs expressed by each cell may comprise the same antigen binding domain, spacer and transmembrane domain and may differ in the intracellular signalling domain. Alternatively, the CARs expressed by each cell may differ in two or more of the CAR components. Alternatively, the CARs expressed by each cell may differ in three or more of the CAR components. Alternatively, the CARs expressed by each cell may differ in all of the CAR components.

The plurality of cells may be obtained using peripheral blood obtained from a single subject or peripheral blood obtained from a number of different subjects. It will be appreciated that the former permits the evaluation of intra-donor variability, while the latter provides consistency.

The second to fifth steps of the second assay of the invention are common with the second to fifth steps of the first assay of the invention. Thus, their definitions, descriptions and particular features and embodiments apply equally to the second assay of the invention.

In a sixth step, the second assay of the invention comprises a step of comparing the expression of genes between sequence sets obtained in step (v).

The term “gene” has been described in detail in the context of the first assay of the invention and its definition, particular embodiments, and examples apply equally to the second assay of the invention.

Optionally, the sixth step comprises identifying the genes from the sets of pooled sequences obtained in the fifth step.

Genes may be identified from the sets of pooled sequences obtained in the fifth step by any conventional method, typically a method of sequence alignment. Methods for the alignment of sequences for comparison are well known in the art and have been described in detail in the context of the first assay of the invention.

The expression of genes is then compared between sequence sets obtained in step (v). This is done by identifying if there are alterations in the expression of the genes, i.e. an increase or decrease of gene expression. The expression of a gene is considered to be increased in one cell, or first cell, compared to another cell, or second cell, when the expression levels in the first cell increase with respect to the expression levels in the second cell by at least 1%, by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more. Similarly, the expression of a gene is considered decreased in the first cell compared to the second cell when the expression levels in the first cell decrease with respect to the expression levels in the second cell by at least 1%, by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by at least 100% (i.e., absent). When the expression levels between the first and second cells is considered not to be altered when the expression levels are increased or decreased by less than 1%, including 0%. It will be appreciated that the same applies when comparing one cell population with another cell population.

Virtually, the change of expression of any gene may provide information about the transcriptional response of each cell expressing a different CAR to a target antigen. By comparing differences in transcriptional response, the skilled person will be able to determine which responses are triggered by the expression of each different CAR. It is also possible to determine the transcriptional effect of each individual CAR component.

Non-limiting examples of genes whose change of expression may provide information about the transcriptional response of a CAR to a target antigen include genes encoding markers of subsets of cells (e.g. naïve T cells, activated T cells, central memory T cells, effector memory T cells, NK cells), genes related to cytokine production (e.g. IL2, IFNγ), genes encoding markers of exhaustion, and other genes encoding markers of activation, proliferation and killing. Specific examples of these genes are provided in the context of the first assay of the invention.

The first and second assays of the invention may be conveniently adapted to test CAR-T cells comprising two or more CARs, such as logic gated CAR-T cells, as would be apparent to a person skilled in the art.

The present invention also contemplates assays according to the first and second assays of the invention, wherein each partition of the plurality of partitions contains a single cell and a unique barcode molecule, wherein each cell comprises a cassette comprising a sequence encoding a chimeric antigen receptor (CAR), wherein each CAR in the partition library are different, that is without the labelling sequence. The identification of each CAR in these assays is carried out by sequencing the antigen-recognising domain or binder. The person skilled in the art will readily be able to adapt the first and second assays of the invention to accommodate this modification in the plurality of partitions.

### 3. Kit

The present invention also contemplates a kit which is suitable for use in the assays of the invention. Thus, in another aspect, the invention provides a kit which comprises a partition library according to the first aspect of the invention and at least one reagent suitable to carry out the assays of the invention.

The reagents suitable to carry out the assays of the invention may be a target cell expressing at least one target antigen specific for the CAR(s) and/or a cell suitable for obtaining the reference sample.

The kit may additionally comprise one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

Instructions for using the kit of the invention according to the assay of the invention may also be provided.

The invention will now be further described by way of Examples, which are meant to serve to assist one of ordinary skill in the art in carrying out the invention and are not intended in any way to limit the scope of the invention.

### EXAMPLES

### Example 1: Detection of Barcode 10 Labelling Sequence at the 5′ UTR of a Cassette Having a Sequence Encoding an Anti-CD19 CAR in Transduced T Cells

A test construct was generated to determine the effect of inserting a 15 bp labelling sequence on the expression of downstream ORF in a cassette having a sequence encoding an anti-CD19 CAR. The labelling sequence, termed Barcode 10 having the sequence shown in SEQ ID NO: 6 (GCTGGCACTACGACA), was derived from Saccharomyces cerevisiae AGA1 gene. The labelling sequence was inserted at different positions in the 5′ UTR of the SFFV promoter, after the predicted transcriptional start site and in 6 bp shifts until it reaches the Kozak sequence.

The construct comprised a 5′ UTR containing the Barcode 10 labelling sequence at different positions (Table 1, below), a sequence encoding the RQR8 marker-suicide gene, and a sequence encoding an anti-CD19 CAR derived from the 4G7 antibody, having a human CD8a stalk spacer and a 4-1BB/CD3zeta endodomain. Each construct was cloned into a pCCL viral expression vector.

TABLE 1
Position of the Barcode 10 labelling sequence
in respect of the Kozak sequence.

| Vector number | Number of bp between Barcode 10 to Kozak sequence |
|---|---|
| 16248 | No barcode |
| 46635 | 23 |
| 46636 | 17 |
| 46637 | 11 |
| 46638 | 5 |

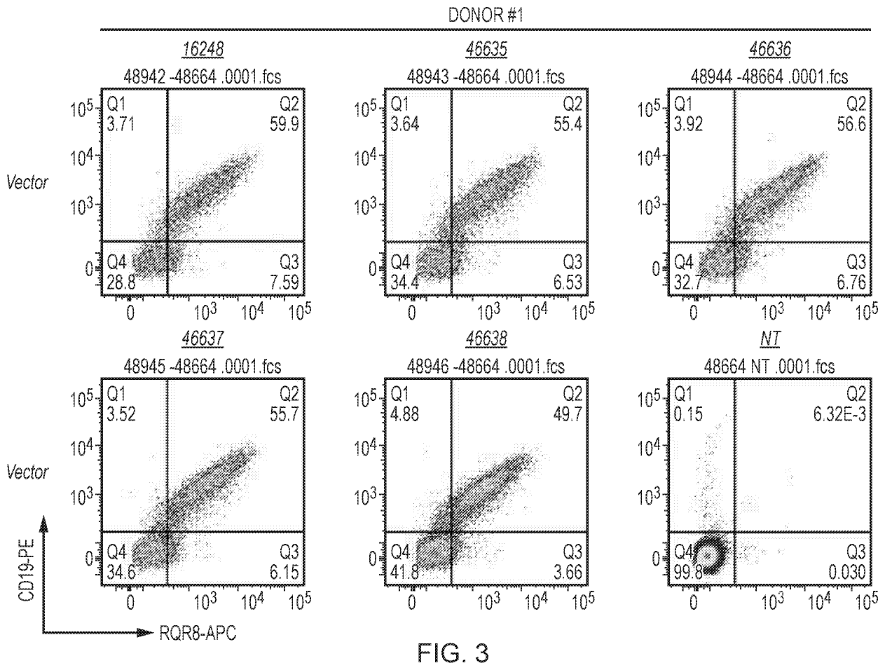

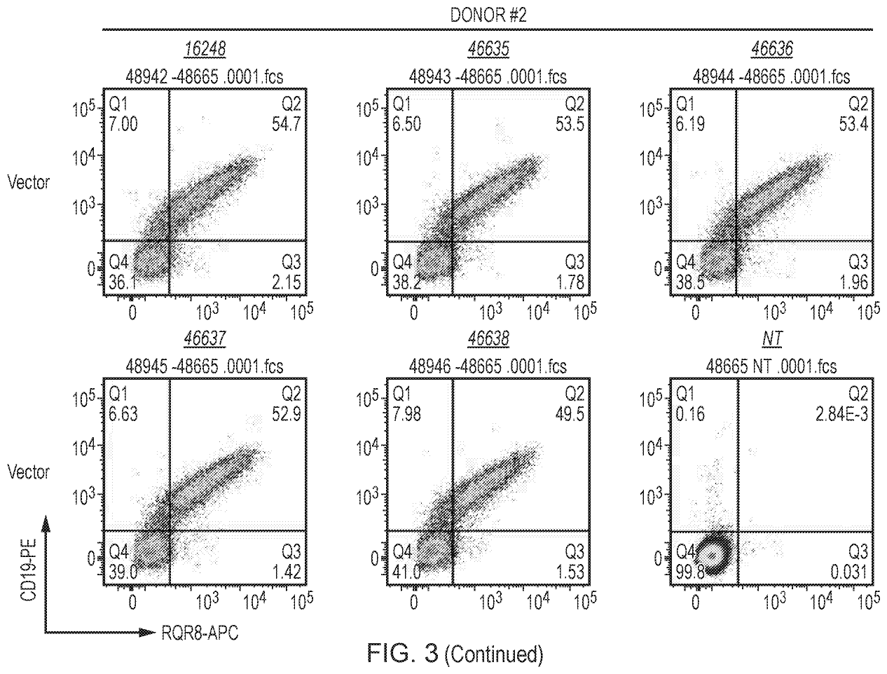

Each vector was used to transduce PBMCs from two donors. All vectors were transduced into PBMCs with similar efficiency, and the expression level of the anti-CD19 CAR was similar among all vectors (FIG. 3).

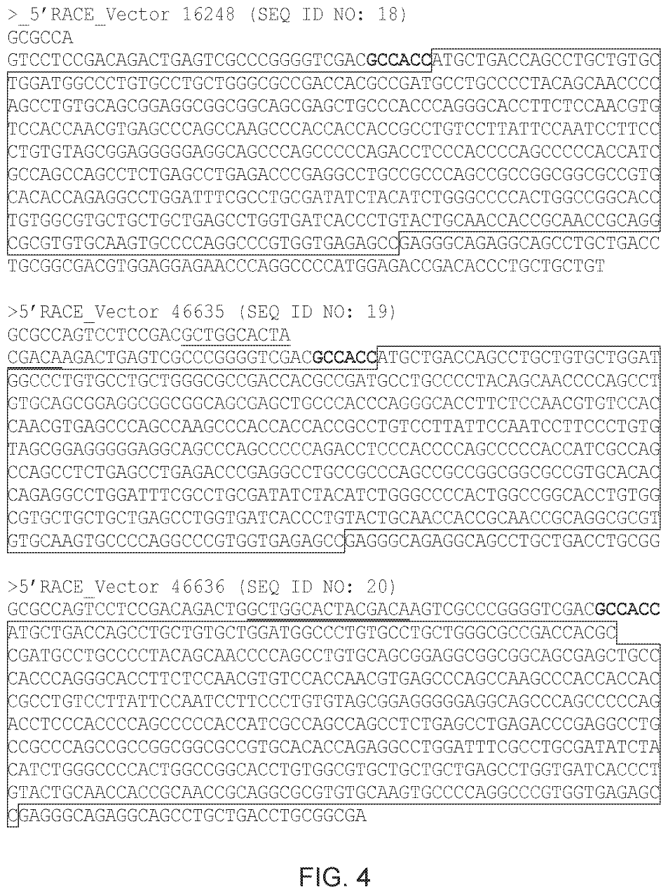

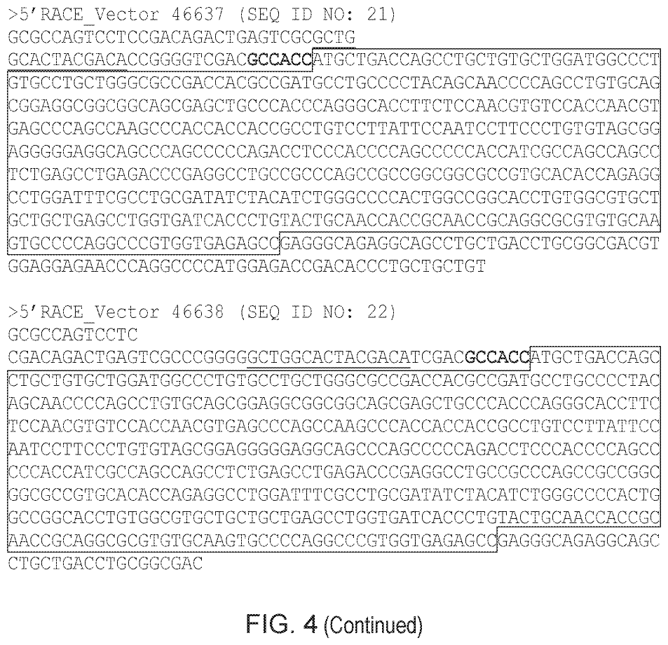

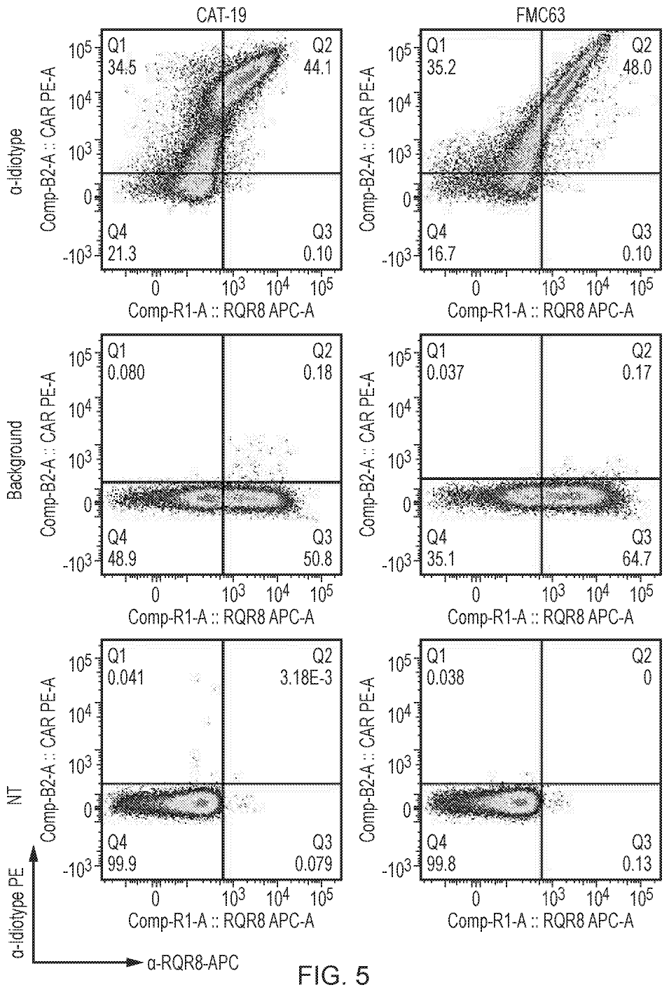

For each construct, the transduced PBMCs from both donors were combined and processed to obtain total RNA. Then, for each construct, 5′ RACE PCR was carried out using reverse primer 5′-ACAGCAGCAGGGTGTCGGTCT-3′(SEQ ID NO: 17), and the PCR product was sequenced using the same primer. Sequencing results revealed that the Barcode 10 sequence is in the right position in the transcript derived from each of the anti-CD29 CAR constructs. FIG. 4 contains the sequencing results from vector numbers 16248, 46635, 46636, and 46638. The sequencing result from vector number 46637 was too short to identify the barcode and was not included.

These results demonstrate that presence of the sequence of Barcode 10 can be detected in the transcript derived from the CAR constructs by 5′ RACE PCR. Moreover, the insertion of a 15 bp labelling sequence in the 5′ UTR does not affect the expression of a downstream ORF in a cassette having a sequence encoding an anti-CD19 CAR.

### Example 2: Screening of the Transcriptome of T Cells Expressing Three Human CD19-Targeting CARs Using a Labelling Sequence Located at the 5′ UTR of the Construct

The constructs of three human CD19 CARs, based on HD37, FMC63, and CAT19 anti-CD19 antibodies were generated in a lentiviral vector. An additional CAR that was based on a non-CD19-recognising antibody, i.e. H5N1, was also generated as a control. All constructs encoded second generation CARs containing a CD8 stalk region, a CD8 transmembrane domain, a 4-1BB co-stimulatory domain, and a CD3ζ signalling domain. A 15 bp labelling sequence specific for each CAR construct was inserted 11 bp upstream of the Kozak sequence in the 5′UTR of the CAR-encoding gene (Table 2).

RQR8 is incorporated into each construct and separated from the CAR by a T2A ribosomal skip sequence. The expression of RQR8 is therefore correlated to CAR expression on the cell surface and served as a marker to determine transduction efficiency.

TABLE 2
Barcode labelling sequences

| CAR | Barcode | | SEQ ID |
|---|---|---|---|
| Construct | ID | Barcode Sequence | NO: |
| HD37 | Barcode 4 | 5′-ATTGCCTTGGCATCT-3′ | 10 |
| CAT-19 | Barcode 5 | 5′-CGATTCTAGTGACGA-3′ | 11 |
| FMC63 | Barcode 6 | 5′-CAAGACAAACGATGC-3′ | 12 |
| H5N1 | Barcode 8 | 5′-GCGCTAGTCTCCACA-3′ | 14 |

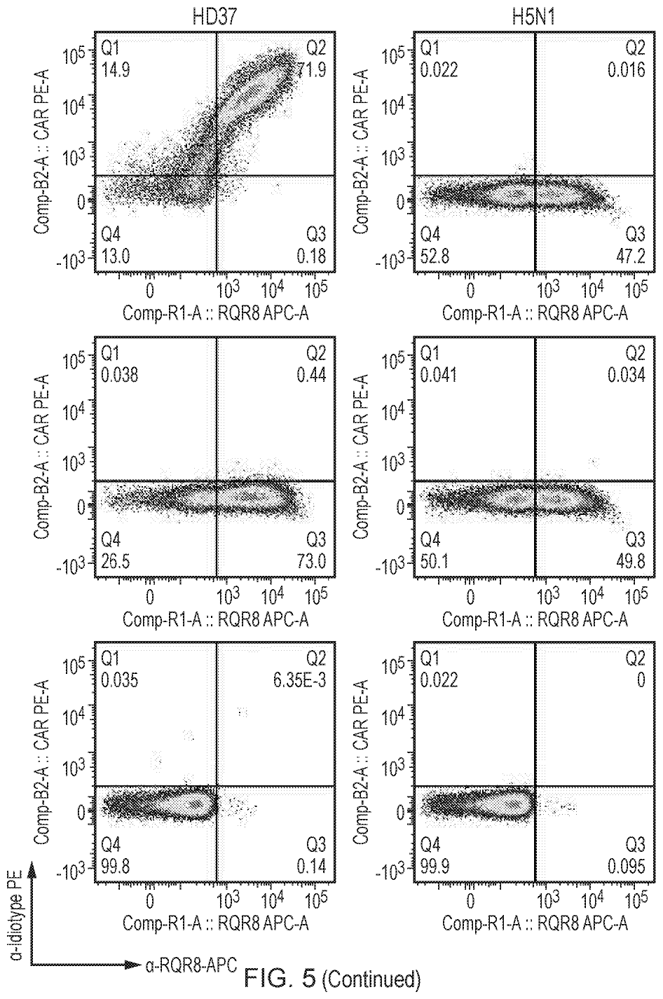

Human peripheral blood mononuclear cells (PBMCs) from two donors at a time were activated by anti-CD3/anti-CD28 co-stimulation in the presence of IL-2. Activated cells were then transduced with lentiviral particles carrying each CAR individually. Transduction efficiency was determined by flow cytometry 96 h following transduction, using fluorochrome-conjugated QBEND10 (to detect RQR8) and an anti-idiotype antibody against each CAR. Cells were then stained with a suitable fluorochrome-conjugated secondary antibody. Representative transduction efficiencies are shown in FIG. 5.

Three cell mixes were set up for T cells expressing each CAR:
a) Without target cell, by incubating the transduced T cells in culture medium only;
b) With a CD19− target cell, by co-incubating the transduced T cells with SupT-1, a T cell line that does not endogenously express CD19
c) With a CD19+ target cell, by co-incubating the transduced T cells with SupT-1 CD19+ cells, which are engineered to express CD19 on the cell surface.

As the T cells were transduced with varying degrees of efficiency, T cell numbers for each CAR was normalized to the CAR T cell compartment with the lowest transduction efficiency using non-transduced T cells from matching donors. This was done to normalize the killing potential between CARs with the highest and lowest transduction efficiencies. Therefore, for (b) and (c), the CAR+ T cell: target cell ratio was set at 1:1.

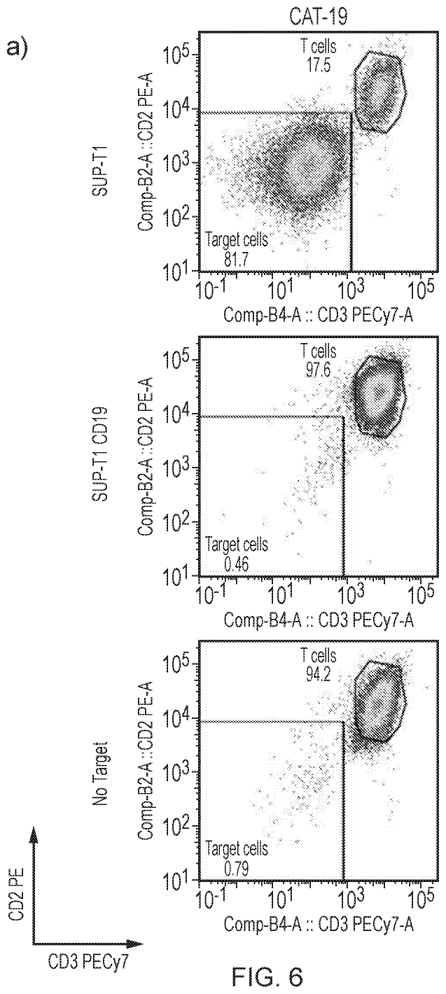

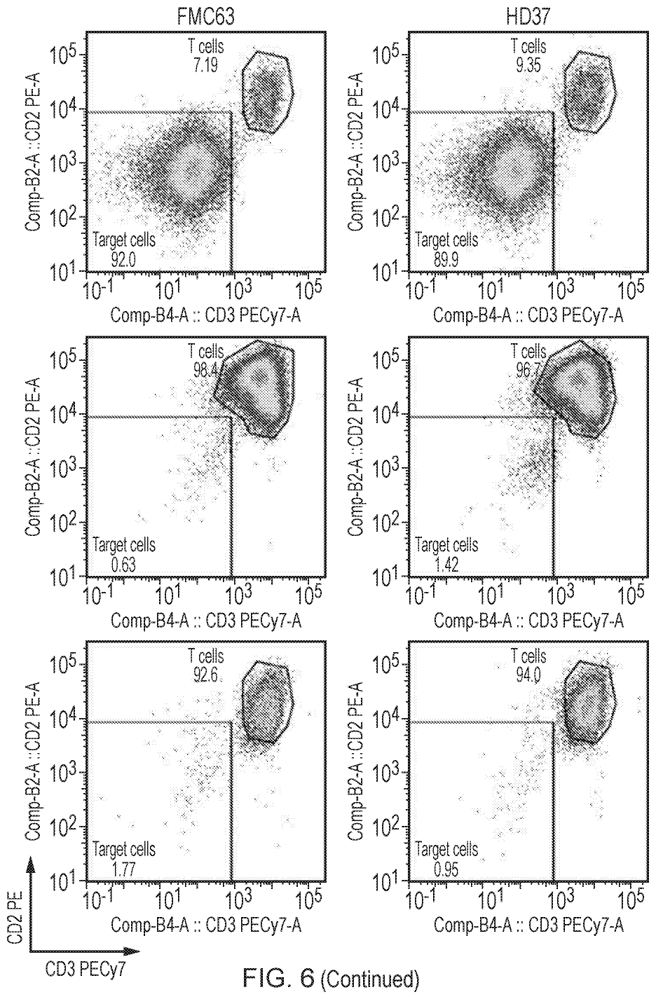

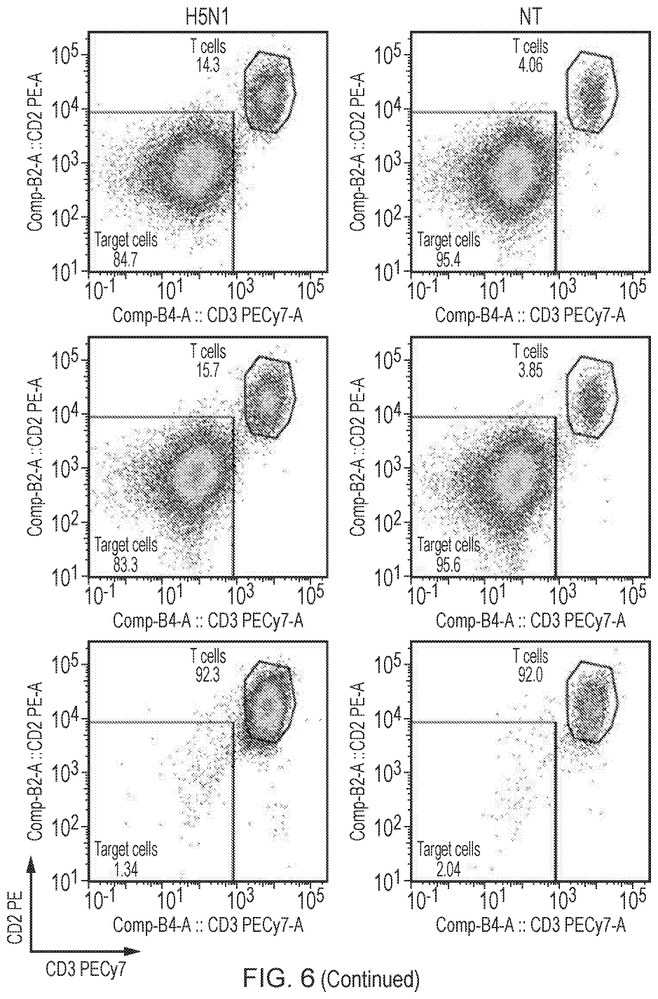

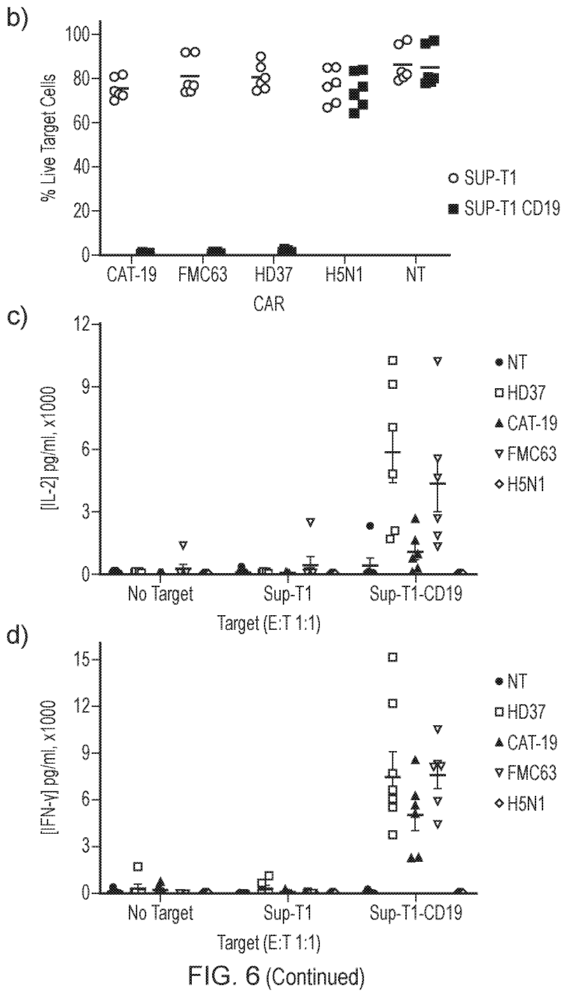

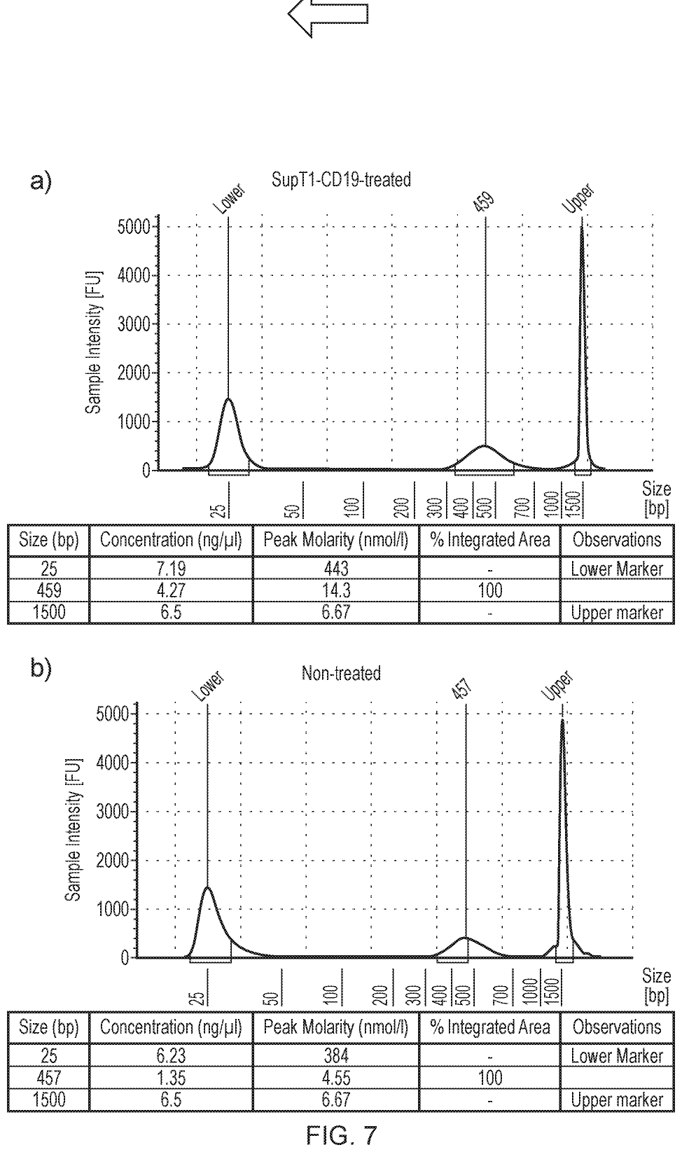

After co-incubation for 72 h, an aliquot of each of cell mixes (a), (b), and (c) was analysed for CD2 and CD3 expression by flow cytometry to confirm that killing had taken place in cell mix (c), but not in cell mix (a) or (b) (FIG. 6a, 6b). The supernatants from these co-cultures were assayed for IL-2 and IFN-γ levels by ELISA. Results shown in FIG. 6c demonstrate that only the T cells expressing anti-CD19 CARs co-cultured with SupT1 CD19+ cells were activated (FIG. 6c).

The remainder cell mixes (a) of all four CAR-expressing T cells were pooled together at a final CAR T cell ratio of 1:1:1:1. The same was done with the remainder cell mixes (c). Any target cells remaining in (c) were depleted by MACS, by staining first with PE-conjugated anti-CD19 followed by anti-PE-conjugated magnetic beads. Dead cells were depleted from (c) by MACS using Annexin V-conjugated magnetic beads.

Groups (a) and (c) of pooled T cells were partitioned into Gel bead in Emulsion (GEM), each containing a single cell. Subsequently, single cell transcriptomes were generated using a scRNA-seq microfluidics platform (10× Genomics). Briefly, the cell mix was counted and 1,000 T cells per group were further diluted into reagents for reverse transcription, which included 30 nucleotide oligo-dT and reverse transcriptase. The diluted cell and reverse transcription reaction mix were mixed with a pool of gel beads each anchored with a unique modified template-switching oligo which comprised, from 5′ to 3′, a sequencing adapter, a unique barcode of 16 nucleotides, a randomised unique molecular identifier (UMI) of 10 nucleotides, followed by a template switching oligo of 13 nucleotides. A single cell and a single gel bead were encapsulated into a GEM on a microfluidic device at the water-oil surfactant interface. Reverse transcription was carried out in each GEM so that each resulting cDNA molecule contained the sequencing adapter, a UMI, and a shared barcode per GEM at its 3′ end.

Subsequently, the emulsion was broken, and all barcoded cDNAs were pooled for cDNA amplification. The barcoded first-strand cDNA from each pool (a) and (c) was purified and amplified by PCR; the amount and quality of the PCR products were assayed using an Agilent Tapestation D5000 chip (FIG. 3). Fifty nanograms of amplified cDNA were fragmented enzymatically and size-selected by solid phase reversible immobilisation-based paramagnetic bead technology (SPRIselect) to the desired fragment approximately 450 bp size prior to library preparation. Briefly, double-size selection was performed by incubating cDNA fragments with the appropriate volumetric ratio of beads, followed by magnetic separation to remove fragments greater than 700 bp and less than 300 bp. 5′ Gene expression libraries were constructed using the digested cDNAs, containing the P5 and P7 Illumina adapter sequences, an Illumina sample index sequence, and an Ilumina read 2 primer sequence. Finally, the libraries were sequenced using the HiSeq 2×150 bp platform (HiSeq 2500 System, Illumina), at a read depth of 3,500 reads per cell.

Finally, the libraries were mixed and sequenced in a single flowcell using the HiSeq 2×150 bp platform, at a read depth of 3,500 reads per cell (Genewiz, South Plainfield, N.J., USA).

### Analysis of Single Cell 5′ RNA-Sea Data

All single cell 5′ RNA-seq data were analysed using 10× Genomics software package Cell Ranger version 3.0.2 on a Linux server. Cell Ranger is a set of analysis pipelines that process the RNA-seq output to align reads, generate feature-barcode matrices based on detected 10× Barcodes associated with single cells and perform clustering and gene expression analysis.

In the current experiment, each CAR is associated with a unique barcode which is located near the 5′ end of corresponding transcript. To facilitate the calling of CAR identity expressed by an individual CAR-T cell, the 120 bp sequence from the transcription start site encompassing 27 bp at the 3′ end of spleen focus forming virus (SFFV) promoter, the 15 bp unique barcode specific for each of HD37-CAR, CAT19-CAR, FMC63-CAR and H5N1-CAR (Table 2), the 17 bp 5′ untranslated region and the 60 bp Human T cell receptor Vβ signal sequence was artificially added into human genome and transcriptome version GRch38 (Genome Reference Consortium Human genome build 38) to create a set of reference genome and transcriptome profiles. Notably, the sequences flanking the CAR-specific barcode as specified above are common to all four CARs. The command ‘mkref’ in Cell Ranger was used to create the reference genomes and transcriptomes.

The bulk sequencing data was demultiplexed by Genewiz using Illumina's bcl2fastq software according to the sample index associated with the individual sequencing library. This yielded two sets of single-cell 5′ RNA-seq data in FASTQ format, one for the pooled CAR-T cells that had not encountered target cell [pool (a)], and the other for pooled CAR-T cells co-incubated with CD19⁺-target cells [pool (c)].

The Cell Ranger ‘count’ pipeline can take FASTQ files and perform single cell sequence analysis including alignment to reference genome and transcriptome profiles, filtering, 10× Barcode counting, and UMI counting. For the current experiment, the specific parameters introduced to Cell Ranger ‘count’ were ‘expecting 1000 cells’ and ‘5’ sequencing chemistry’. Each set of FASTQ data was fed into Cell Ranger ‘count’ separately. After running the analysis, a Quality Control (QC) report including estimated number of cells, mean sequence reads per cell, and mean identified genes per cell was issued for individual dataset (Table 3). Further, through aligning with the reference genome and transcriptome profiles, individual CAR-T cells expressing HD37-CAR, CAT19-CAR, FMC63-CAR or H5N1-CAR were identified unambiguously from either dataset. Through UMI counting of gene expression reads, the gene expression profile of individual cell was also generated.

TABLE 3
Summary of 10X 5′ RNA-seq data

| | Esti- | Mean | | | Number | Number | |
|---|---|---|---|---|---|---|---|
| | mated | se- | | Number | of | of | Number |
| Cell | num- | quence | Median | of HD37 | FMC63 | CAT19 | of H5N1 |
| popu- | ber of | reads | Genes | CAR-T | CAR-T | CAR-T | CAR-T |
| lation | cells | per cell | per cell | cells | cells | cells | cells |
| Pooled | 1,002 | 165,191 | 2,820 | 80 | 34 | 62 | 52 |
| CAR-T | | | | | | | |
| cells | | | | | | | |
| w/o | | | | | | | |
| target | | | | | | | |
| cells | | | | | | | |
| Pooled | 1,020 | 215,720 | 2,116 | 150 | 71 | 76 | 20 |
| CAR-T | | | | | | | |
| cells | | | | | | | |
| co- | | | | | | | |
| incu- | | | | | | | |
| bated | | | | | | | |
| with | | | | | | | |
| CD19⁺ | | | | | | | |
| target | | | | | | | |
| cells | | | | | | | |

Cell Ranger ‘aggr’ is a pipeline used to aggregate the output of ‘count’ generated from pools (a) and (c) into one result file with sample attributes defined for each cell including: the experiment setup (co-incubated with/without CD19⁺-target cells), whether the cell is the remaining CD19⁺-target cell, whether the cell is expressing CAR, and if expressing, the identity of the CAR. The type of file produced by ‘aggr’ bears the extension ‘cloupe’. Subsequently, 10× software Loupe Cell Browser 3.1.0 was used to process the ‘cloupe’ file. Through this software, the 5′ gene expression profiles of individual CAR-T cells were extracted from the entire dataset for further analysis. The Loupe Cell Browser allows the identification of significant genes by comparing the expression profiles among different cell population, and clustering of cell types based on their unique gene expression profiles. This software also generates tSNE plot using a dimensionality reduction algorithm to graphically visualize the separation of very large datasets, such as the separation of cell clusters.

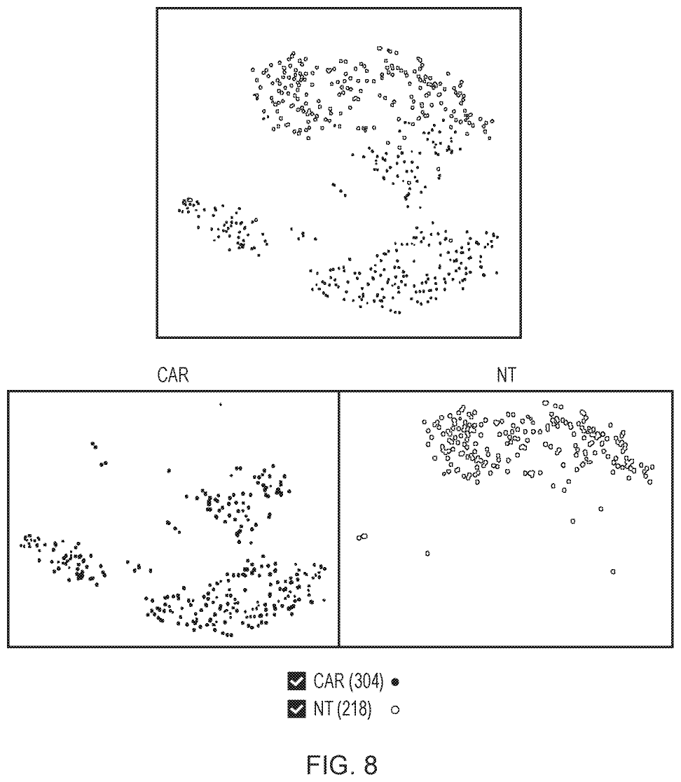

The results obtained revealed clear separations of cell populations in cell clusters. For example, the tSNE plot shown in FIG. 8 visualises cell clustering of CAR-expressing T cells with CD19 stimulation versus CAR-expressing T cells without the stimulation, showing a clear separation. This reflects the significant difference in gene expression profiles across a diverse spectrum of genes in response to stimulation by the target.

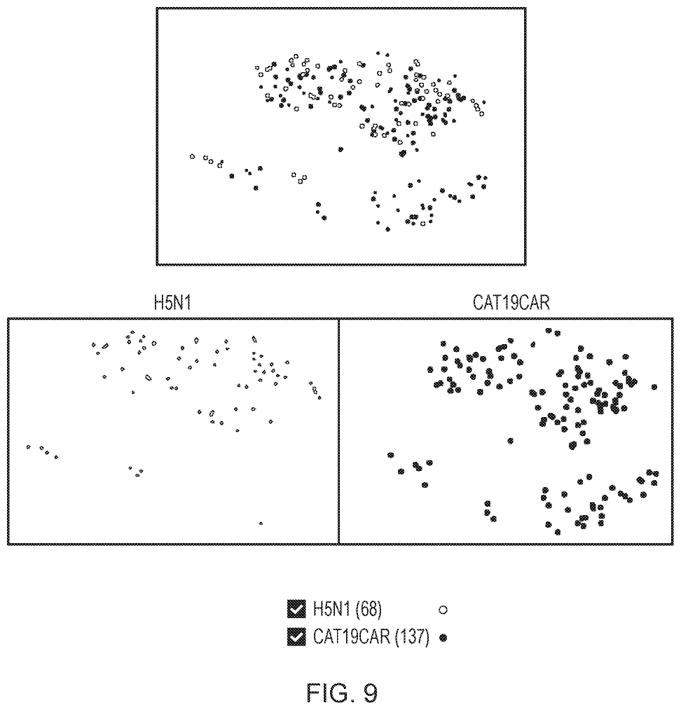

A similarly clear separation was achieved when comparing cells transduced with H5N1 CAR (H5N1) vs cells transduced with anti-CD19 CARs, all co-cultured with CD19⁺⁻ target cells (FIG. 9). This suggests the change in gene expression profile associated with anti-CD19 CARs is derived from specific binding between the CARs and CD19 target and the concomitant signalling events, and not from non-specific binding and signalling.

Overall, these results demonstrate that labelling different CAR constructs with unique barcodes to screen and profile CAR-T cells with single cell RNA-Seq is a successful strategy. Furthermore, these unique barcodes are useful in detecting the functional differences between different CAR-T cell populations. In conclusion, the methods described herein are useful for identifying CARs with desirable properties in an automated manner.

1. A partition library comprising a plurality of partitions, wherein each partition contains a single cell and a unique barcode molecule, wherein each cell comprises a cassette comprising a sequence encoding a chimeric antigen receptor (CAR) and a labelling sequence, wherein each CAR and each labelling sequence in the partition library are different.

2. The partition library according to claim 1, wherein the labelling sequence is located in the 5′ untranslated region (UTR) of the sequence encoding the CAR.

3. The partition library according to claim 1, wherein the labelling sequence is located in the sequence encoding the signal peptide of the sequence encoding the CAR.

4. The partition library according to claim 1, wherein the labelling sequence is located in the 3′ UTR of the sequence encoding the CAR.

5. The partition library according to any of claims 1 to 4, wherein the labelling sequence comprises at least 5 bp.

6. The partition library according to any of claims 1 to 5, wherein each cassette further comprises a second sequence encoding a second CAR.

7. The partition library according to claim 6, wherein each cassette further comprises a third sequence encoding a third CAR.

8. The partition library according to any of claims 1 to 7, wherein the cassettes are DNA or RNA.

9. The partition library according to any of claims 1 to 8, wherein the cells are cytolytic immune cells.

10. The partition library according to claim 9, wherein the cytolytic immune cells are T cells or NK cells.

11. The partition library according to any of claims 1 to 10, wherein the cells are incubated with a target cell expressing a target antigen.

12. An assay for analysing the transcriptional response of a CAR to a target antigen, which comprises the following steps:
(i) providing a plurality of partitions according to claim 11;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) identifying genes within a given set which are differentially expressed by the cell following exposure to target antigen.

13. The assay according to claim 12, wherein step (vi) identifies at least one gene selected from the group consisting of a gene related to cytokine production, a gene encoding a marker of naïve T cells, a gene encoding a marker of activated T cells, a gene encoding a marker of central memory T cells, a gene encoding a marker of effector memory T cells, a gene encoding a marker of exhaustion, a gene encoding a marker of activation, a gene encoding a marker of proliferation, and a gene encoding a marker of cell killing.

14. An assay for comparing the transcriptional responses of a plurality of cells to a target antigen, which comprises the following steps:
(i) providing a plurality of partitions according to claim 11, the cell in each partition expressing a different CAR against the same target antigen;
(ii) performing reverse transcription such that all RNA sequences in the cell within the partition are barcoded with the unique barcode molecule;
(iii) disrupting the partitions and pooling the barcoded nucleic acid sequences from (ii);
(iv) sequencing the pooled sequences;
(v) analysing the pooled sequences to find sets of sequences with the same unique barcode; and
(vi) comparing the expression of genes between sequence sets.

15. The assay according to claim 14, wherein step (vi) further identifies at least one gene selected from the group consisting of a gene related to cytokine production, a gene encoding a marker of naïve T cells, a gene encoding a marker of activated T cells, a gene encoding a marker of central memory T cells, a gene encoding a marker of effector memory T cells, a gene encoding a marker of exhaustion, a gene encoding a marker of activation, a gene encoding a marker of proliferation, and a gene encoding a marker of cell killing.

16. Kit comprising a partition library according to any of claims 1 to 11, and at least one reagent suitable to carry out the assay according to any of claims 12 to 15.

17. The kit according to claim 15, further comprising one or more components selected from the group consisting of partitioning fluids, barcode molecule libraries, which may be associated or not with microcapsules, reagents for disrupting cells, and reagents for amplifying nucleic acids.

18. The kit according to any of claim 16 or 17, further comprising instructions for using the kit in the assay according to any of claims 12 to 15.


SEQUENCE LISTING (22 sequences)

SEQ ID NO: 1 - protein, 20 aa, Artificial Sequence
  2A-like cleavage site sequence
RAEGRGSLLTCGDVEENPGP

SEQ ID NO: 2 - protein, 21 aa, Artificial Sequence
  signal peptide
MGTSLLCWMALCLLGADHADG

SEQ ID NO: 3 - protein, 21 aa, Artificial Sequence
  signal peptide derived from IgG1
MSLPVTALLLPLALLLHAARP

SEQ ID NO: 4 - protein, 20 aa, Artificial Sequence
  signal peptide derived from CD8
MAVPTQVLGLLLLWLTDARC

SEQ ID NO: 5 - RNA, 7 nt, Artificial Sequence
  Kozak consensus sequence
accaugg

SEQ ID NO: 6 - DNA, 15 bp, Artificial Sequence
  labelling sequence
gctggcactacgaca

SEQ ID NO: 7 - DNA, 15 bp, Artificial Sequence
  labelling sequence
gacattatctttcgc

SEQ ID NO: 8 - DNA, 15 bp, Artificial Sequence
  labelling sequence
cattttacctacctg

SEQ ID NO: 9 - DNA, 15 bp, Artificial Sequence
  labelling sequence
cacaatattgttggg

SEQ ID NO: 10 - DNA, 15 bp, Artificial Sequence
  labelling sequence
attgccttggcatct

SEQ ID NO: 11 - DNA, 15 bp, Artificial Sequence
  labelling sequence
cgattctagtgacga

SEQ ID NO: 12 - DNA, 15 bp, Artificial Sequence
  labelling sequence
caagacaaacgatgc

SEQ ID NO: 13 - DNA, 15 bp, Artificial Sequence
  labelling sequence
caactacagtttcac

SEQ ID NO: 14 - DNA, 15 bp, Artificial Sequence
  labelling sequence
gcgctagtctccaca

SEQ ID NO: 15 - DNA, 15 bp, Artificial Sequence
  labelling sequence
tccactatcgttcaa

SEQ ID NO: 16 - DNA, 6 bp, Artificial Sequence
  polyadenylation signal sequence
aauaaa

SEQ ID NO: 17 - DNA, 21 bp, Artificial Sequence
  reverse primer
acagcagcagggtgtcggtct

SEQ ID NO: 18 - DNA, 601 bp, Artificial Sequence
  5′ RACE_Vector 16248
gcgccagtcctccgacagactgagtcgcccggggtcgacgccaccatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcggcgacgtggaggagaacccaggccccatggagaccgacaccctgctgctgt

SEQ ID NO: 19 - DNA, 566 bp, Artificial Sequence
  5′ RACE_Vector 46635
gcgccagtcctccgacgctggcactacgacaagactgagtcgcccggggtcgacgccaccatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcgg

SEQ ID NO: 20 - DNA, 569 bp, Artificial Sequence
  5′ RACE_Vector 46636
gcgccagtcctccgacagactggctggcactacgacaagtcgcccggggtcgacgccaccatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcggcga

SEQ ID NO: 21 - DNA, 616 bp, Artificial Sequence
  5′ RACE_Vector 46637
gcgccagtcctccgacagactgagtcgcgctggcactacgacaccggggtcgacgccaccatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcggcgacgtggaggagaacccaggccccatggagaccgacaccctgctgctgt

SEQ ID NO: 22 - DNA, 570 bp, Artificial Sequence
  5′ RACE_Vector 46638
gcgccagtcctccgacagactgagtcgcccgggggctggcactacgacatcgacgccaccatgctgaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcggcgac